SEQUENCE LISTING (22 sequences)

SEQ ID NO: 1 - DNA, 33 bp, Artificial Sequence
  Description of the artificial sequencePrimer
aaatgagctcaaatgtatatgtatggaaaaacc

SEQ ID NO: 2 - DNA, 33 bp, Artificial Sequence
  Description of the artificial sequencePrimer
aatgagctcgaaacgatatcttcaaaaaaaaac

SEQ ID NO: 3 - DNA, 33 bp, Artificial Sequence
  Description of the artificial sequencePrimer
aaactggatccagaacaatctaacgctttctcg

SEQ ID NO: 4 - DNA, 31 bp, Artificial Sequence
  Description of the artificial sequencePrimer
atataggatcctcaaccagauaucacaggtg

SEQ ID NO: 5 - DNA, 29 bp, Artificial Sequence
  Description of the artificial sequencePrimer
aaatatctagagatctcaaatcgacgacc

SEQ ID NO: 6 - DNA, 29 bp, Artificial Sequence
  Description of the artificial sequencePrimer
atataggatcccattttacctcgatggac

SEQ ID NO: 7 - DNA, 28 bp, Artificial Sequence
  Description of the artificial sequencePrimer
aatgagctctgtgtcacctgctagatcc

SEQ ID NO: 8 - DNA, 29 bp, Artificial Sequence
  Description of the artificial sequencePrimer
atataggatccagatatcacaggtgaaac

SEQ ID NO: 9 - DNA, 33 bp, Artificial Sequence
  Description of the artificial sequencePrimer
ataggatccaggagcagaagtcccaaggcaaag

SEQ ID NO: 10 - DNA, 32 bp, Artificial Sequence
  Description of the artificial sequencePrimer
aaagtcgacagaaggtagaggagatcttgatc

SEQ ID NO: 11 - DNA, 31 bp, Artificial Sequence
  Description of the artificial sequencePrimer
aaactaagcttggtatcttcttccctgcaag

SEQ ID NO: 12 - DNA, 37 bp, Artificial Sequence
  Description of the artificial sequencePrimer
aaacctaggcggctactcagctgatacctcagagcag

SEQ ID NO: 13 - DNA, 34 bp, Artificial Sequence
  Description of the artificial sequencePrimer
aaactaagcttaaatattgaaccggcctcggttc

SEQ ID NO: 14 - DNA, 37 bp, Artificial Sequence
  Description of the artificial sequencePrimer
aaactggatcctcttcctgttggtcgtcgacgatttg

SEQ ID NO: 15 - DNA, 29 bp, Artificial Sequence
  Description of the artificial sequencePrimer
aaactggatcctcttcctttatcaaatcc

SEQ ID NO: 16 - DNA, 30 bp, Artificial Sequence
  Description of the artificial sequencePrimer
atataccatgggtagccgatggaatcgtac

SEQ ID NO: 17 - DNA, 24 bp, Artificial Sequence
  Description of the artificial sequencePrimer
atataccatgggcagtcgttcgag

SEQ ID NO: 18 - DNA, 4044 bp, Unknown
  Description of the unknown organismgenome atSRp30
agacaaagatgcttacttcttaaacatgttcgaggtttattgaaaatgatcaccagcttctaactatggtatcttcttccctgcaagcgaacagtggaagatgattgataacggaaatatcggaacatcactcaacaaaccaaaaatttggacatcatatcgcaacaaattcaataggaaaaatactgaaattccaaaacagaaaaaccaaacggaacagagcaggaactcacggactgagagagaccgtggacggtgtcacggcggaaaatggtcttggacggagttactaatcggcgaattgagatttgagaggtggtagtagaggaaccgagagaatgtttctctcaaaaaaatccccaagtgtttccgatctagtgtctcttttgtccaaaaacgacagtgtttaggaaacctaggagaatgaatgacccgatggtccgaatccgattcgaaattggttcgaattgtaataactaacatacaatattccggtttgaatgataagaaaaaacacattcgatccggttagaacaatatattaacaggcccattaaaacatatgggccgatcttgatcaactgggctattcatcgttgatacatgcggccgcacaggattaaaatccagttccgttttataaaaggatactagtttccaaacgaacggtggttgtctcctttccagaacaatctaacgctttctcgaacatcttcttcttcttctttctcgaaattatttttccagtaatcaatttcttctcttctagatttttacaggaactaattttctgctctgaggtatcagatgagtagccgatggaatcgtacgatctacgttgggaatttgcctggagatattcgcaagtgtgaggttgaagatctcttctacaaggtttgaaaatttcctcttttctctcgataaaaattgaattcattatgactagtttgggttcataaatttgcaattctgtcttgctgagacaatttaaatcgactcttatgtatatttgtttcagtatggaccaattgtggacattgatttgaagattccaccgagacctcctggttatgcctttgtcgaggtatattgatcaagtacaaatttgtttttttcttcttcttgtaatagtataggctaatgactaagatagtttgttattggtggcagtttgaagatcctcgtgatgcagacgatgcaatttatggacgtgatggttatgattttgatgggtgtcgacttcgggttagtaaacgcatgatgaaagctagcttaattttctgtaatttcttgtaaaggtgttatctttgtgtgatgtttttaggttgagattgcacatggtggtcgtagattttcaccatcagttgataggtacagcagcagctacagtgcgagccgtgcaccttcaagacgctctgactaccgcggtttgtagagtcttctcgattgtgttatttggtgttgtgtaaaattttatatttgaaaactcatttttactacctaaacatgtagtgcttgtgaccggattaccgccttctgcttcgtggcaggaccttaaggtaagggacactatatagtctttttctctgaatgttggttctctatatcatgttttggatttatctcttttctgaaatgatgttatttgctatttacgggtgattaggatcacatgcgcaaagctggagatgtctgcttctctgaagttttccctgaccgtaaaggtgagttgacattcgatagtttggataagctttttgattgatgtgttagtaaattagtctttgtgaaggagaataggtgttaagcatctgaactgctaaactcacattcagtatttctttgtaggcatgtctggggttgtggattatagcaactatgatgatatgaagtacgcagtaagttttatatctttgcaacgcaaatgttcctggacttatgccttagactgcttttgtttcatagtataccgagctgaatttatcttcctggaggccagtgttggatctttgatgttcccttaaaatttttgatgtgacagataaggaaacttgatgccactgaatttcgaaatgctttctctagtgcttatatacgggtatgttgtattgctttctttgattttgttaagcataagtggatatggagtcatctctgaatttactgttcaggtgagggaatatgagtcgaggagtgtgagtcgaagcccagatgattctaaaagctatagaagcaggagtcggagccgtggtccaagctgtagctatagtagcaagagcaggaggtctttttttttttttttttttcataaacctaagacatataagggatttttattgtaacttatttatgaaattaactgacttctaaatgcaatgcagtgtgtcacctgctagatccatttccccgcgttcacggccccttagtcgttctcgctcgctatacagctctgtctcaaggtatgagtgttagatttgtatcattattatatatgtagttaccccttcatggatcacttgttcttgcatagtgaactccttactagctttattacttacaactaagcaccttttgttgcttccgtacacagttgaatttgtttgagtcttttttccctcatagtggactagtctattgtcacttgattttcttcctttgttgatgttttctatgtcatgcaaactccaatatgggtaaaggttacctccttgtttgggattaccagagttccttttcatttcttacacgtgaatgtgtttgttttttatgttttgagttcttgacagagatgctcccatcatatttagtccttttcctttctctttgtgtcgttctcttctggatgtttccttctgataaagctttacttcttaacttttttccagcgacggtgaatttattacgtatcaacctcaatatccgacctataatttaaagaacacttagctagatgttcacttttgaaaatttatttctatcggaagggggcagatgatttctgaggcatgtcctcgatcatttaccgtgattacaattgtattgcgttgtttgtttctagatctggctcactgctacgagctggggattggatctagatgggtcatctagatggattcttggactggatttacaaagctggattagcatgaactgaacttctgttttacggtctggtctggtctggtactccgcgcgtatcagctgtaggatctgatcgcaaagttttggactatgattactctgattcctcaatatatttatctttttgacaatagtggattctgtgttgagttcttttctaggacagcatttaagctcccgggactagatgggagatggtcagtaaatttctttgttatgccacacttacatggggtttttcggtcttgctgcaggtcccaatcaagatcaaaatcaagatcaagatcaagatcgaattctccagtttcacctgtggtaagtctaaaagctgaaccttctttaattcacaatccatgtgtttgtttaaatacctgctcactttggttgttcttcaatcaacaccaacttaacgaaatcatgagacagactataaaatttgaagagtctgtagaacgactaggtctcaccaacctctgtgtgcactaaaaatcgcctctccaagtgtttcagcaacataatctacctctgtcatgtgttatcatttcttcttctcttaacggtattacatattatgttttgcaggtgatatctggttgaaaatgaaaactggccactggctgtacccgaatcgtctcaagcttctcaggctccactgctaatagaatttgattccgatttgggattattatactggtcttcttgtatgggacgaccaatatgtctttctagttttagttgtgaacctggaattggtctgttattgtgtcattaaaaagccggaaactctgtctcggctgcataataaagttcatcagacattgtgttgggtgtggtgaggtttttccatacatatacatttacattacaactactggtgtcttttatgattatcttaaactaaac

SEQ ID NO: 19 - protein, 279 aa, Unknown
  Description of the unknown organismgenome atSRp30
MSSRWNRTIYVGNLPGDIRKCEVEDLFYKYGPIVDIDLKIPPRPPGYAFVEFEDPRDADDAIYGRDGYDFDGCRLRVEIAHGGRRFSPSVDRYSSSYSASRAPSRRSDYRVLVTGLPPSASWQDLKDHMRKAGDVCFSEVFPDRKGMSGVVDYSNYDDMKYAIRKLDATEFRNAFSSAYIRVREYESRSVSRSPDDSKSYRSRSRSRGPSCSYSSKSRSVSPARSISPRSRPLSRSRSLYSSVSRSGSLLRAGDWISQSRSKSRSRSRSNSPVSPVISG

SEQ ID NO: 20 - DNA, 1132 bp, Artificial Sequence
  Description of the artificial sequence atSRp34/SR1
aggagcagaagtcccaaggcaaagtcttcacgtaggtcccctgcaaaatctacatcaagatctcctggcccccgctcgaagtcaaggtcaccgtctccaagaaggtaatgatttgattccttttcagaatgcaccaggcaacattacttagtcggtagattttcctgtgaagtttatgaaaatctgattagaaaagtcattgttgatgctcaaagttcttgttgattgagcatatactggttttttgttgacacctgctgtttgctcgtcattattgtcccttcttactccatccttatatttagatgcttagcccttttctggttaagatttgcgagattggccttacatttttagcatttttaaatatgctcttttctttcctgagaagaaaatggttttgtttcatagatatggatttacatacgactagaaaatggaaattaatatctggatggaaattgattgttgacaagtgtttcgtctaagaggtatgggaaactttggaatagagactttgcttttcgtggcttcctgatatagtattcactaatttacattgctgctagatggataacagtggagacattggatcacttggatcacaatattatatcgggatttctgtaaaactatattggctcgatggattgacaatatggaatctgggctctcttgggacgtacgtggctcatttggcaacacaagtttttttcgccacatggcttataaaacctctgtcctatcacctatgttttaactaagtagcagaatagtttggtttatgttccttttttttatttgttgcaacttcttaatctctgtgagatagaaggagaggctccaggaccttgctgaacagtataaaacacaacatgtttggatttttgaatctgagtttcttttcttggacttttgcagatcgcgttcaagatcaagatctcctctaccttctgtgagtaacaagatccaacttgtgacccctcttttattttgacataatcttctgttttacattgttcgttatcttaatagcttttctgtatcacaggttcagaaggaaggaagcaagagccctagcaagccaagtccagccaagagtcctatccacactaggagtccatcgaggtaa

SEQ ID NO: 21 - protein, 78 aa, Artificial Sequence
  Description of the artificial sequence atSRp34/SR1
RSRSPKAKSSRRSPAKSTSRSPGPRSKSRSPSPRRYGFTYDSRSRSRSPLPSVQKEGSKSPSKPSPAKSPIHTRSPSR

SEQ ID NO: 22 - protein, 248 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

(19) United States
(12) Patent Application Publication
(10) Pub. No.: US 2002/0115180 A1
(43) Pub. Date: Aug. 22, 2002

(54) Splicing factor

(75) Inventors: Andrea Barta, Rannersdorf (AT); Sergyi Lopato, Wien (AT); Maria Kalyna, Wien (AT); Silke Dorner, Wien (AT)

Correspondence Address: Mark B. Wilson, FULBRIGHT & JAWORSKI L.L.P., Suite 2400, 600 Congress Avenue, Austin, TX, 78701, US

(73) Assignee: Osterreichisches Forschungszentrum Seibersdorf Gesellschaft M.B.H., Vienna (AT)

(21) Appl. No.: 10/014,927

(22) Filed: Oct. 23, 2001

Related U.S. Application Data
(63) Continuation of application No. PCT/AT00/00100, filed on Apr. 20, 2000.

(30) Foreign Application Priority Data
Apr. 23, 1999 (AT) A 727/1999

(51) Int. Cl. C12N 9/22; A01H 1/00; C12P 21/02; C12N 5/04

(52) U.S. Cl. 435/199; 435/410; 435/320.1; 435/69.1; 800/278

(57) ABSTRACT

Disclosed is a protein having a splicing factor activity in plants, which

comprises the amino acid sequence of the protein according to FIG. 1A, or

comprises the sequence of the amino acids 1 to 4, 7 to 19, 45 to 52, 111 to 116, and 149 to 153 of the protein according to FIG. 1A and has more than 85% similarity with this protein, or

comprises more than 60% similarity with the splice proteins atSRp34/SR1 and SF2/ASF according to FIG. 2, wherein the G-rich sequence, which corresponds to the amino acids 85 to 113 of the atSRp34/SR1 protein is substituted by an S-rich sequence, or

corresponds to, or is derived from, the protein corresponding to FIG. 1A from a plant other than Arabidopsis thaliana.

The invention relates to proteins comprising splicing factor activity in plants.

Alternative pre-mRNA-splicing is part of the expression program of a large number of genes in animals and plants. It allows the selection of different combinations of splice sites within a given pre-mRNA, generating structurally and functionally distinct protein isoforms (Breitbart et al. 1987; Manley and Tacke 1996; Cáceres and Krainer, 1997). Several protein factors involved in the regulation of alternative splicing have been described, including a family of RNA-binding proteins containing arginine/serine-rich regions (SR proteins) (for reviews, see Fu 1995; Chabot 1996; Valcarcel and Green 1996; Cáceres and Krainer 1997). SR proteins are highly conserved nuclear phosphoproteins that are members of a protein family and share a serine phospho-epitope recognized by the monoclonal antibody mAb104 (Roth et al. 1991). They consist at least of one RNA-binding domain (RBD) [the typical RBD or RNA recognition motif (RRM) domain of approximately 80 amino acids] (Birney et al. 1993), and possess several serine/arginine (SR) dipeptides near their carboxy termini (Zahler et al. 1992). In general, SR proteins are a defined subgroup of a large superfamily of nuclear proteins with RS-rich domains of variable sequence and position (Fu 1995).

The human SF2/ASF splicing factor, a prototype of an SR protein, is essential for the first cleavage step in pre-mRNA splicing (Krainer et al. 1990b; Ge et al. 1991) and can also determine in a concentration-dependent manner which 5′ splice site is selected in pre-mRNAs containing alternative sites (Ge and Manley 1990; Krainer et al. 1990a). The preferential usage of the proximal 5′ splice site at higher concentrations of SF2/ASF is counteracted by members of the hnRNP A/B family of proteins (Mayeda and Krainer 1992; Mayeda et al. 1994), suggesting that the relative abundance of these and possible other antagonistic splicing factors determines the patterns of alternative splicing in vitro and in vivo (Mayeda and Krainer 1992; Cáceres et al. 1994; Yang et al. 1994; Wang and Manley 1995; Hanamura et al. 1998). SF2/ASF and any other SR protein can complement splicing-deficient S100 extracts that essentially lack SR proteins, but individual SR proteins sometimes display distinct specificities and efficiencies in splicing different pre-mRNAs (Krainer et al. 1990b; Ge et al. 1991; Fu and Maniatis 1992; Kim et al. 1992; Mayeda et al. 1992; Fu 1993; Zahler et al. 1993; Screaton et al. 1995; Wang and Manley 1995). Furthermore, an important activity of SF2/ASF and other SR proteins is the interaction with exonic enhancer sequences, which stimulate usage of an adjacent weak splice site (Sun et al. 1993; Tian and Maniatis 1993; Staknis and Reed 1994; Ramchatesingh et al. 1995; Tacke and Manley 1995; Lou et al. 1996). The RNA-binding specificity of an SR protein is conferred by the RRM region and adjacent sequences (Cáceres and Krainer 1993; Zuo and Manley 1993; Tacke and Manley 1995; Allain and Howe 1997; Tacke et al. 1997); the various RS domains are responsible primarily for protein-protein interactions, which are modulated by the phosphorylation status of these regions (Wu and Maniatis 1993; Amrein et al. 1994; Kohtz et al. 1994; Zuo and Maniatis 1996; Xiao and Manley 1997). In addition, it has been demonstrated that RS domains modulate the RNA-binding activity and subnuclear localization of SR proteins (Li and Bingham 1991; Hedley et al. 1995; Cáceres et al. 1997). Recently, SF2/ASF, SRp20, and 9G8 have been shown to shuttle between the nucleus and the cytoplasm, and this property depends on the presence and type of RS domain (Cáceres et al. 1998).

The studies described above led to the proposal that SR proteins might function by bridging splice sites through RNA-protein and protein-protein interactions, therefore establishing early interactions for splice-site definition and for the assembly of spliceosomes. These interactions are stimulated by binding of SR proteins to nearby enhancer sequences and modulated by phosphorylation/dephosphorylation of the RS regions.

There is limited information about the regulation of SR protein expression in vivo. In the case of SF2/ASF and SRp20, significant differences in mRNA and protein levels have been observed in various cell types and tissues (Jumaa et al. 1997; Hanamura et al. 1998). SC35 expression is also highly variable in cell lines (Fu and Maniatis 1992; Vellard et al. 1992) and some SR proteins are activated by mitogens (Diamond et al. 1993; Screaton et al. 1995). Interestingly, several alternatively spliced SR protein mRNAs have been described, which code for truncated proteins of still unknown function (Ge et al. 1991; Cavaloc et al. 1994; Screaton et al. 1995; Jumaa et al. 1997). Human SRp20 autoregulates its expression at the level of splicing by binding to its own pre-mRNA, thereby preventing overexpression of the active protein (Jumaa and Nielsen 1997). It was found recently that the amounts of alternatively spliced mRNAs coding for SRp30b and SRp20 in Caenorhabditis elegans may be regulated at least in part at the level of mRNA stability (Morrison et al. 1997).

Hitherto, in organisms comprising a protein corresponding to the human SF2/ASF protein, merely one variant each of this protein has been found.

Little is known about the mechanisms of intron excision in plant cells. It is generally assumed that the basic mechanism of splicing in plants is similar to that of yeast and mammals (Solymosy and Pollak 1993; Luehrsen et al. 1994; Filipowicz et al. 1995; Brown and Simpson 1998). Nevertheless, animal introns are not processed in any plant tissue so far examined (Barta et al. 1986; Van Santen and Spritz 1987; Wiebauer et al. 1988). Apparently, the process of intron recognition differs in these two kingdoms. One of the determining features of introns in plants seems to be a U or AU-rich character, whereas the exons are more GC-rich (for review, see Brown and Simpson 1998). As SR-proteins play a critical role in correct splice-site selection in mammals, it is of interest to characterized the corresponding protein family in plants. When screened for similar proteins in plants by using the mAb 104 antibody or a specific monoclonal antibody raised against human SF2/ASF, it could be demonstrated that plants do possess SR proteins, including SF2/ASF-like proteins (Lopato et al. 1996a). However, the plant SR proteins are of different size and are smaller than 55 kD. Furthermore, it could be shown that plant SR proteins are active in constitutive and alternative splicing when assayed in heterologous HeLa cell extracts.

In an effort to isolate individual plant splicing factors, arginine/serine-rich proteins from Arabidopsis belonging to two different families have been characterized (Lopato et al. 1996b; 1999). Both families show good homology in the amino-terminal RRM with animal SR proteins, but at their carboxy-terminal domains they are richer in arginine than in serine, have few SR dipeptides, and they were therefore termed RS proteins. Nevertheless, these proteins are recognized by the mAb104 antibody and can complement SR protein-deficient HeLa S100 extracts efficiently.

In Arabidopsis thaliana, a protein with similarity to human SF2/ASF protein could be identified (Lazar et al. 1995) which is known under the name of SR1, atSRp34 or atSRp34/SR1.

Influencing the splicing process in plants is one of the possible ways of arriving at improved plants. Therefore, it is an object of the present invention to provide new plant splicing factors which can be used within the scope of biotechnology.

According to the invention, this object is achieved by a protein having splicing factor activity in plants, which either

comprises the amino acid sequence of the protein according to FIG. 1A of the appendix, or

comprises the sequence of the amino acids 1 to 4, 7 to 19, 45 to 52, 111 to 116, and 149 to 153 of the protein according to FIG. 1A of the appendix and has more than 85% similarity with this protein, or

comprises more than 60% similarity with the splice proteins atSRp34/SR1 and SF2/ASF according to FIG. 2 of the appendix, wherein the G-rich sequence, which corresponds to the amino acids 85 to 113 of the atSRp34/SR1 protein is substituted by an S-rich sequence, or

corresponds to, or is derived from, the protein corresponding to FIG. 1A from a plant other than Arabidopsis thaliana.

The protein according to the invention advantageously is characterized in that it comprises the sequence of the amino acids 1 to 4, 7 to 19, 22 to 72, 74 to 85, 96 to 141, 149 to 153, 156 to 172, with amino acid 168 being variable, yet not D or N, of the protein according to FIG. 1A of the appendix. The highly conserved sequences indicated preferably are arranged such that the distances between the sequences indicated approximately correspond to those according to FIG. 1A of the appendix, preferably not more than 5 amino acid deletions or amino acid insertions being provided. Particularly preferred are sequences in which either no, or merely one single, deletion or insertion is present between the conserved sequences.

The protein according to the invention may comprise one or more amino acid substitutions as compared to the protein illustrated in FIG. 1A of the appendix, as long as the sequence of the amino acids 1 to 4, 7 to 19, 45 to 52, 111 to 116, and 149 to 153 of the protein according to FIG. 1A of the appendix or the function of the protein as splicing factor are not affected. Its function as a splicing factor is to be considered to be affected if the protein comprises, e.g., only 10 to 20% of the natural activity of the protein according to FIG. 1A of the appendix.

Amino acid substitutions may particularly be provided in the region of the amino acids of FIG. 2 of the appendix not marked as conserved, in particular if they do not essentially influence the three-dimensional structure or the charge distribution of the protein. In these regions, e.g., an interchange of hydrophobic amino acids or an interchange of aromatic amino acids is possible, unless the natural activity is lost thereby.

According to the invention, all proteins are preferred which comprise an amino acid sequence having at least 90% identity with the sequence of the amino acids 1 to 85 and 96 to 222 of the protein according to FIG. 1A of the appendix.

Proteins which comprise an amino acid sequence that has at least 95%, in particular at least 98%, identity with the sequence of the amino acids 1 to 85 and 96 to 222 of the protein according to FIG. 1A of the appendix are particularly preferred.

With the proteins of the invention it has been possible for the first time to find a second SF2/ASF-similar protein in an organism (adjacent atSRp34/SR1), resulting in the possibility of a combined influencing of the splice process. In the appendix it is shown that the two proteins mutually influence the splicing process but also have an effect on other proteins which, moreover, with the appropriate genetic control, will result in surprising possibilities of changing the splicing properties of the plant or of the plant cell.

The S-rich sequence between amino acid 95 and 102 of the sequence according to FIG. 1A of the appendix preferably comprises at least 4, in particular at least 7, serine residues. Advantageously, it is G-free, in particular free from GGR or GGR repeats.

The protein according to the invention can be provided by standard methods of molecular biology also from other plants, because of the high homology of such proteins, such as by PCR coupled cloning methods or by other screening of gene libraries, e.g. with the sequence according to FIG. 1A of the appendix.

According to a further aspect, the present invention relates to nucleic acid molecules, which either

comprise a nucleic acid sequence according to FIG. 1A of the appendix, or

comprise a nucleic acid sequence encoding a protein of the invention, or

comprise a nucleic acid sequence which, under stringent conditions, binds to the nucleic acid molecule according to FIG. 1A and encodes a splice protein active in plants or is complementary thereto.

By stringent conditions, e.g., hybridizing in 7% SDS, 0.5 M NaPO₄, pH 7.0, 1 mM EDTA at 50° C. followed by washing with 3% SDS is to be understood.

These molecules may either be DNA or also RNA molecules. In the case of RNA molecules, the alternative spliced forms, in particular those described in the appendix, are preferred embodiments of the present invention.

Another object of the invention is a biologically functional vector which is characterized in that it comprises a nucleic acid molecule according to the invention.

In a further aspect, the invention moreover relates to a system comprising a nucleic acid which encodes a protein according to any one of the claims, and a nucleic acid which encodes

the atSRp34/SR1 protein of Arabidopsis thaliana or

the protein corresponding to FIG. 1A from a plant other than Arabidopsis thaliana, or

a protein derived from these proteins, wherein at least one of the nucleic acids is under the control of a promoter not naturally connected with these nucleic acids.

By atSRp34/SR1 -derived proteins also those proteins are to be understood which define the proteins according to the invention, starting from the protein illustrated in FIG. 1A, in analogous manner.

Preferably, both nucleic acids are controlled by promoters which are not naturally connected with these nucleic acids.

Preferably, at least one of the promoters under whose control the nucleic acids are is an inducible promoter.

According to a preferred embodiment of the present invention, the nucleic acid encoding a protein according to the invention is under the control of a promoter which causes an overexpression of this protein.

Preferably, the nucleic acid which encodes a protein according to the invention is under the control of a promoter which, under defined conditions, prevents the expression of this protein and, under defined different conditions, enables the expression of this protein.

According to a further aspect, the present invention relates to transgenic plants or plant cells which express a protein according to the invention and comprise a nucleic acid molecule, a vector or a system according to the invention.

Moreover, the present invention relates to the use of a protein according to the invention, a nucleic acid according to the invention, a vector according to the invention or a system according to the invention for changing the splice properties of a plant cell or of a plant.

Moreover, the subjects of the invention may be used for changing the development behavior of a plant.

A particular aspect lies in the use for retarding the flowering stage of plants.

According to the invention, it has been shown that by such a use, a retardation of the flower formation of a plant of at least 15%, preferably at least 25%, as compared to its wild-type can be induced, e.g. if the protein according to the invention is put under the control of a strong promoter.

Particularly preferred plants within the scope of the present invention are economic crop plants, such as cereal, beans, rice or fruit plants.

The invention will now be explained in more detail by way of the following examples as well as the drawing figures, yet it shall not be restricted thereto.

### IN THE FIGURES

FIG. 1A shows a genomic sequence of at SRp30 ; promoter and intron sequences are indicated in lower-case, cDNA sequences in upper-case, and the TATA box in bold italics. The derived protein sequence is shown below the DNA in the one-letter code. The bold sequence in the tenth intron is contained in alternative spliced mRNAs, and the underlined sequence is included if both cryptic 3′ and 5′ splice sites are used. The conserved RNP submotifs of both RRMs are boxed. The open arrows mark the ends of promoter sequences used for expression studies. Solid horizontal arrows indicate the ends of the PCR product obtained with degenerate primers and used as a probe for library screening. (↓) The end of the Gat SRp30 clone. The sequence data of the at SRp30 gene was submitted to the EMBL database (accession number AJ131214).

FIG. 1B shows a schematic representation of Gat SRp30 , its mRNA isoforms, and derived proteins. Exons are shown as boxes and introns as lines (bold lines: introns included in the alternatively spliced mRNAs). Exonic 5′ and 3′ untranslated regions are shaded, and the coding regions are black. (*) The new stop codon in the alternatively spliced products.

FIG. 1C shows a schematic representation of Gat SRp34, its mRNA isoforms, and derived proteins. The drawings are as in B (accession number AF001035).

FIG. 1D shows a partial genomic sequence of atSRp34/SR1 starting from exon 10 up to the stop codon. The bold sequence in the long intron is included in alternatively spliced mRNAs, the underlined sequence is included when both cryptic 3′ and 5′ splice sites are used.

FIG. 2 shows the alignment of Arabidopsis at SRp30 , atSRp34/SR1, and human SF2/ASF protein sequences (solid area). Positions at which a single residue occurs in at least two of the sequences (shaded areas). Conservative substitutions such as RK, IVL, ED, FY, ST. The positions of the conserved RNP-1 and RNP-2 submotifs and the glycine-rich region are indicated.

FIG. 3 shows the expression of at SRp30 and atSRp34/SR1 in wild-type Arabidopsis thaliana. (A) Expression in different plant tissues. Northern blot analysis of poly(A)⁺RNA from leaves (L), stems (S), roots (R), and flowers (F) is shown. One microgram of RNA was loaded per lane, and the blots were either probed with a probe corresponding to the tenth intron of Gat SRp30, or with the cDNAs of at SRp30 or atSRp34/SR1. (B, C) Developmental expression of at SRp30. Total RNA was isolated from whole plants on different days during development starting from the day of germination. The RNA was either used for Northern blot analysis with 10 μg/lane of total RNA and the membrane probed with at SRp30 cDNA (B), or for analysis of the RT-PCR products on a 1.2% agarose gel (C). The two primers for the PCR reaction were located in the exons adjacent to the tenth intron.

FIG. 4 shows the immunodetection of phosphorylated and dephosphorylated SR proteins from cell cultures of Arabidopsis. (A) Immunodetection of proteins in SR protein preparations (lanes 1-4). (Lane 1) Monoclonal antibody mAb104, which is specific for a common phosphoepitope of SR proteins; (lane 2) polyclonal antibody raised against recombinant at SRp30; (lane 3) polyclonal antibody raised against recombinant atSRp34/SR1; (lane 4) monoclonal antibody specific for human SF2/ASF. Protein markers are indicated at the left. (B, C) Dephosphorylation of SR proteins with alkaline phosphatase. (Lanes 1, 2) SR proteins incubated at 37° C. without enzyme for 0 and 24 h, respectively; (lanes 3, 4) SR proteins incubated with 0.2 and 1.0 u/μl of enzyme for 3 h; (lanes 5, 6) SR proteins incubated with 1.0 and 2.0 u/μl of enzyme for 24 h. Proteins were probed either with anti-p30 (B) or anti-P34 (C).

FIG. 5 shows the transcriptional analysis of at SRp30 (A-D) and atSRp34/SR1 (E-H) promoter-GUS fusions in transgenic Arabidopsis. GUS staining of (A, E) flowers at the postanthesis stage; (B, F) staining patterns in the leaves; (C, G) primary and developing lateral roots; (D, H) seedlings (2 days after germination).

FIG. 6 shows the overexpression of at SRp30 in transgenic Arabidopsis plants. The cDNA and the genomic sequences of at SRp30 were cloned under the control of the strong 35S CaMV promoter (constructs pC30 and pG30, respectively) and used to transform Arabidopsis plants. (A) Northern blot analysis of total RNA isolated from independent transgenic lines probed with at SRp30 cDNA. (Lanes 1-4) Plants transformed with the pG30 construct; (lanes 5-9) plants transformed with pC30; (lane 10) control plant transformed with pBI121 (35S CaMV-GUS construct). mRNA isoforms are indicated. (B) RT-PCR analysis of the same transgenic lines. The primers used are indicated with arrows in the diagram of Gat SRp30. (Lane 10) Four times more material was loaded in this control lane. (C) Immunodetection of at SRp30 in total protein extracts of overexpressing transgenic plants. (Lane 1) SR protein preparations from Arabidopsis cell culture (wild type); (lane 2) line transformed with pG30; (lanes 3-7) lines transformed with pC30; (lane 8) pC30 transgenic line that does not overexpress mRNA1. Antibody raised against at SRp30 (anti-p30) was used for immunodetection.

FIG. 7 shows phenotypic changes in plants overexpressing at SRp30. (A) Plants overexpressing at SRp30; (B) wild-type plants; (C) plants overexpressing at SRp30 grown under short day conditions displays no apical dominance, resulting in a bushy phenotype.

FIG. 8 shows the regulation of the alternative splicing pattern of atSRp31 and U1-70K in plants overexpressing at SRp30. The relevant alternative splicing events are shown in the diagrams at the top. RT-PCR was carried out using primers (marked with arrows) derived from the adjacent exons. (A) Changes in splicing in the second intron of GatRSp31. (Lane 1) Control line transformed with pBI121 (35SCaMV-GUS construct), (lanes 2-5) lines transformed with the pG30 construct; (lanes 6-10) lines transformed with pC30. (B) Correctly spliced mRNA encoding U1-70K protein is produced upon overexpression of at SRp30. (Lane 1) control plant transformed with pBI121 (35SCaMV-GUS construct), (lanes 2, 3) lines transformed with the pG 30 construct; (lanes 4-8) plants transformed with pC30; (lane 9) control pC30 transgenic plant that does not overexpress mRNA1; (lane 10) 19-day old wild-type plant.

FIG. 9 shows changes in the expression pattern of atSRp34/SR1 in plants overexpressing at SRp30. (A) Preferential usage of an alternative 5′ splice site in the eleventh intron of atSRp34/SR1. The gene, the relevant alternative splicing events, and the derived proteins are depicted at the top, the gel showing the RT-PCR products obtained with primers from the adjacent exons. (Lane 1) Control, untransformed wild-type plant; (lanes 2 to 5) lines transformed with the pG30 construct; (lanes 6 to 11) plants transformed with pC30; (lane 11) control pC30 transgenic plant that does not overexpress mRNA1. (B) Immunodetection of atSRp34 and atSRp34s in total protein extracts from transgenic plants. (Lanes 1, 2) SR protein preparations isolated from Arabidopsis plants and cell culture, respectively; (lane 3) control plant transformed with pC30 that does not overexpress mRNA1; (lane 4) line transformed with pC30; (lane 5) line transformed with the pG30 construct. Anti-p34 antibodies were used for immunodectection.

### EXAMPLES

### MATERIALS AND METHODS

### Purification of SR-proteins and Protein Sequencing

The SR protein purification has been described previously (Lopato et al. 1996a). The proteins were separated by SDS-PAGE. After Coomassie G staining, the protein bands were excised and subjected to in-gel digestion with Lys-C endopeptidase. The resulting peptides were separated by HPLC and sequenced by automated Edman degradation as described previously (Wang et al. 1996).

### Isolation and Sequencing of Genomic DNA and cDNA of at SRp30 and atSRp34/SR1

Total DNA of carrot, tobacco, and Arabidopsis was isolated by the cetyltrimethylammonium bromide (CTAB) method (Murray and Thompson 1980). The most conserved part of the sequenced peptide YVGNLPGDI was used to design two degenerate forward primers. Two reverse degenerate primers were derived from the peptide sequence SWQDLKDHM, which was also part of a sequenced peptide. All four combinations of degenerate primers were used in PCR reactions with genomic DNA from different plants as a template. PCR products were subcloned and sequenced. The PCR product from Arabidopsis was used as a probe for screening a genomic λZAPII library of a. thaliana var. Columbia (Stratagene). One positive clone, Gat SRp30 , was found, mapped, subcloned, and sequenced (EMBL accession number AJ131214). A partial cDNA sequence was found by a BLAST search through the EST databases with the accession number R 65514, 4018 Arabidopsis thaliana cDNA clone 17J1T7, and was kindly provided by the Arabidopsis Biological Resource Center at Ohio State University. Sequence homology was analyzed using Genetics Computer Group (GCG, University of Madison, Madison, Wis.) sequence analysis software package version 7.1-UNIX. Sequencing of the cDNA and the genomic clones was done using an A.L.F. DNA Sequencer and Auto Read Sequencing kit (Pharmacia). To obtain a cDNA clone of atSRp34/SR1, the EST clone with the accession number ACT76795, 11573 atcDNA clone 151I9T7, was sequenced. The genomic sequences for atSRp34/SR1 (accession number AF001035) were isolated by PCR and sequenced.

### Analysis of Alternatively Spliced Isoforms

RNA blot analyses were done as described (Lopato et al. 1996b). cDNAs encoding alternatively spliced isoforms were obtained by RT-PCR using total RNA preparations from A. thaliana plants at different stages of development and primers derived from the 3′ untranslated region (for reverse transcription).

(1) 5′-AAATGAGCTCAAATGTATATGTATGGAAAAACC-3′ (at SRp30 ) and

(2) 5′-AATGAGCTCGAAACGATATCTTCAAAAAAAAC-3′ (atSRp34/SR1)

(The underlined nucleotides correspond to restriction sites) and from the beginning of the 5′ untranslated region at the end of coding region for (PCR):

(3) 5′-AAACTGGATCCAGAACAATCTAACGCTTTCTCG-3′ (at SRp30 ) and

(4) 5′-ATATAGGATCCTCAACCAGAUAUCACAGGTG-3′ (at SRp30 ); and

(5) 5′-AAATATCTAGAGATCTCAAATCGACGACC-3′ (atSRp34/SR1) and

(6) 5′-ATATAGGATCCCATTTTACCTCGATGGAC-3′ (atSRp34/SR1).

All products were sequenced. Alternative splicing of the long introns was studied using primers derived from adjacent exons:

(7) 5′-AATGAGCTCTGTGTCACCTGCTAGATCC-3′ (at SRp30 ) and

(8) 5′-ATATAGGATCCAGATATCACAGGTGAAAC-3′ (at SRp30 ); and

(9) 5′-ATAGGATCCAGGAGCAGAAGTCCCAAGGCAAAG-3′ (atSRp34/SR1) and

(10) 51′-AAAGTCGACAGAAGGTAGAGGAGATCTTGATC-3′ (atSRp34/SR1).

The fragments were analysed on a 1.2% agarose gel.

### Constructs for promoter analysis and overexpression

Approximately 1 kb of promoter sequences of at SRp30 and atSRp34/SR1 plus their complete 5′ untranslated regions (including the first intron in the case of atSRp34/SR1) were obtained by PCR from genomic clone Gat SRp30 and genomic DNA of A. thaliana, respectively, as a template, using primers

(11) 5′-AAACTAAGCTTGGTATCTTCTTCCCTGCAAG-3′ (at SRp30 );

(12) 5′-AAACCTAGGCGGCTACTCAGCTGATACCTCAGAGCAG-31 (at SRp30 );

(13) 5′-AAACTAAGCTTAAATATTGAACCGGCCTCGGTTC-3′ (atSRp34/SR1);

(14) 5′-AAACTGGATCCTCTTCCTGTTGGTCGTCGACGATTTG-3′ (atSRp34/SR1); and

(15) 5′-AAACTGGATCCTCTTCCTTTATCAAATCC-3′ (atSRp34/SR1)

containing HindIII and BamHI restriction sites. These fragments were digested with HindIII and BamHI and fused to the GUS reporger gene (Jefferson 1987) in pBI101 (Clontech).

The cDNA and the genomic sequences of atSRp30 were amplified from cDNA and genomic DNA from A. thaliana using primers 3 and 1, containing BamHI and SacI restriction sites, respectively. The DNA fragments were sequenced and inserted into the BamHI-SacI restriction sites of the pBI121 (Clontech) vector (with the GUS gene deleted) under the control of the 35S CaMV promoter, giving rise to constructs named pC30 and pG30, respectively.

### Cultivation of Plants and Suspension Cultures and Plant Transformation Procedure

Conditions for maintaining carrot and tobacco BY2 suspension cultures were as described earlier (Lopato et al. 1996a). The suspension culture of A. thaliana ecotype Columbia was a gift from Czaba Koncz (Max-Planck-Institut fur Züchtungsforschung, Köln, Germany) and was maintained as described in Lopato et al. (1999). A. thaliana ecotype Columbia was used in all transfer experiments. Seeds were surface-sterilized by a 30 min imbibition with water, followed by a 1 min incubation in 70% ethanol, 10 min in 10% bleach and five rinses with sterile water. Sterilized seeds were grown in MS medium (Murashige and Skoog 1962), supplemented with 1% sucrose. Plants were usually maintained in 16 h light/8 h dark cycle at 23° C., unless otherwise indicated. The cell culture was grown in medium containing Murashige and Skoog salts, 2×Gamborg's vitamins (Gamborg et al. 1968), 1 mg/liter 2,4-dichlorophenoxy-acetic acid (2,4-D), and 3% (wt/vol) sucrose. Cell cultures were incubated in 50 ml of medium in 250 ml conical flasks on a rotary shaker at 110 rpm at 23° C. in dim light. Cell suspensions were subcultured every 7 days and were diluted threefold with fresh medium at each subculture.

All constructs were introduced into Agrobacterium tumefaciens LBA4404 (Hoekema et al. 1983), using a triparental mating procedure with the helper plasmid pRK2013 (Ditta et al. 1980). Root explants of A. thaliana were transformed using the method described by Valvekens et al. (1988) with one modification: Root explants were taken from 15-day-old seedlings grown on vertically placed agar plates (Huang and Ma 1992). Seeds of transgenic plants were germinated in the presence of 50 μg/ml of kanamycin.

### Histochemical Assay of GUS Expression

Histochemical assays of GUS expression were carried out with 5-bromo-4-chloro-3-indolyl glucuronide (X-Gluc, Duchefa) as a substrate and were performed on intact seedlings or excised organs of mature plants grown in vivo as described by Jefferson (1987). The samples were treated with 70% ethanol for 2-6 h to remove chlorophyll from tissues where necessary.

### Purification of SR Proteins from Arabidopsis and Immunoblotting

SR proteins were purified from 3-week-old Arabidopsis plants or 5-day-old suspension cultures using a two-step salt precipitation method as described (Lopato et al. 1996a). Total protein extracts were prepared with the buffer for SR protein isolation (Zahler et al. 1992).

Proteins from the magnesium precipitate and total protein extracts were separated on a 12.5% SDS gel. Protocols for immunoblotting and detection have been described earlier (Lopato et al. 1999). Protein dephosphorylation was carried out with alkaline phosphatase from calf intestine (Biolabs).

### Expression of at SRp30 and atSRp34/SR1 in Bacteria

The coding region of at SRp30 cDNA was amplified by PCR using the primers 5′-ATATACCATGGGTAGCCGATGGAATCGTAC-3′ and (4).

The coding region of atSRp34/SR1 cDNA was amplified by PCR using the primers 5′-ATATACCATGGGCAGTCGT-TCGAG-3′ and (6). The primers contain NcoI and BamHI restriction sites. To obtain the NcoI restriction site, the fourth nucleotide of the coding region in both cases was changed to G. Thus expressed at SRp30 and atSRp34/SR1 have Ser2-Arg and Ser2-Gly substitutions, respectively. The fragments were cloned into the bacterial expression vector pET-3d (Novagen) and transformed into the E. coli strain BL21(DE3)pLysS (Novagen).

Single colonies were grown in 100 ml of LB medium to a density of 0.4-0.5 OD⁵⁵⁰, transferred to 900 ml of fresh pre-warmed medium and incubated for 1 h. The cultures were induced with 0.4 mM IPTG and were grown for a further 5 h. The bacterial pellet was washed twice in 500 ml of ice-cold washing buffer (100 mM NaCl, 10 mM Tris-HCl at pH 7.5) and used for inclusion body isolation: 2 grams of bacterial pellet was resuspended in 18 ml of ice-cold buffer A (10 mM Tris-HCl at pH 7.9, 100 mM KCl, 2 mM DTT, 35% sucrose). 4.5 ml of ice-cold buffer B (333 mM Tris-HCl at pH 8.0, 100 mM EDTA at pH 8.0, 40 mg of lysozyme) was added and incubated on ice for at least 10 min. Lysis buffer (22.5 ml, 1 M LiCl, 20 mM EDTA, 0.5% NP-40) was added, and the solution was sonicated for a total of 2 min at full power (15 sec bursts with cooling periods of 1 min in between). Following centrifugation for 10 min (Sorvall, SS34, 13000 rpm), the pellet was washed twice in 25 ml of buffer C (10 mM Tris-HCl at pH 8.0, 0.1 mM EDTA, 0.5 M LiCl, 0.05% NP-40, 1 mM DTT), and then twice with the same buffer lacking LiCl. The isolated inclusion bodies contained >95% pure protein and were used for polyclonal antibody preparation (Lopato et al. 1999).

### RESULTS

### Isolation and Sequencing of Genomic and cDNA Clones of at SRp30

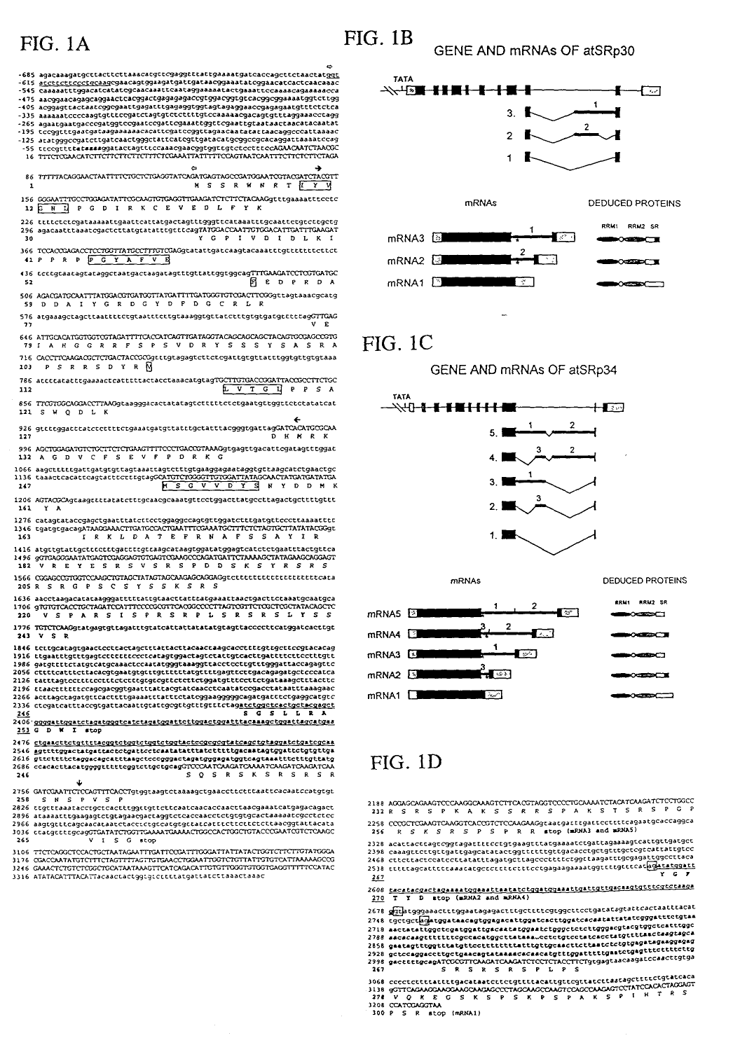

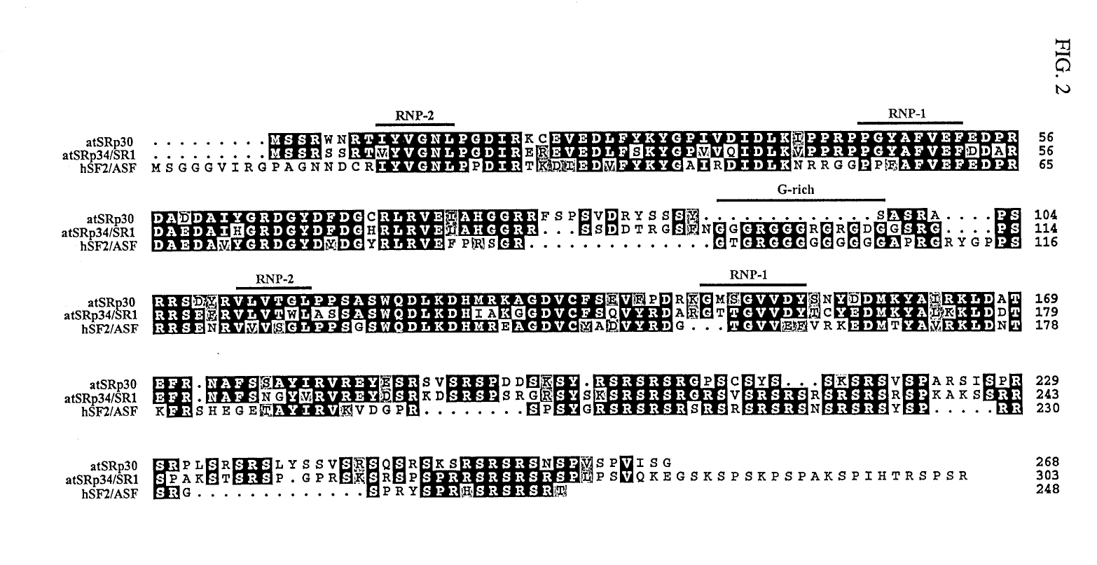

Initially, plant SR proteins in magnesium chloride pellets from protein extracts of carrot and tobacco cell cultures and from Arabidopsis plants were identified. The proteins were detected with the monoclonal antibody mAb104, specific for a shared phosphoepitope in all SR proteins, and with a monoclonal antibody specific for the human SF2/ASF splicing factor (Lopato et al. 1996a). A protein of approximately 50 kD from the carrot SR preparation, which was recognized by both antibodies, was isolated and partially sequenced. Two peptides with significant homology to human SF2/ASF and atSRp34/SR1 were found. The first peptide comprised the RNP-2 submotif of the first RRM, and the second peptide had high homology to a sequence situated between RNP-2 and RNP-1 of the second RRM (see Materials and methods; FIG. 2, above). Based on these sequences, degenerate primers were synthesized and used for PCR on purified genomic DNA from carrot, tobacco, and Arabidopsis. Sequencing of the cloned PCR products revealed two very homologous sequences from Arabidopsis and tobacco. The Arabidopsis fragment was 838 bp long and contained five introns. The fragment borders of the Arabidopsis fragment are marked with black arrows in FIG. 1A. The protein sequence derived from the PCR sequence had extensive homology to both human SF2/ASF and atSRp34/SR1 (FIG. 2). The PCR product from Arabidopsis was used as a probe to screen a λ ZAPII genomic library of A. thaliana. One genomic clone was found and designated Gat SRp30 (genomic clone of Arabidopsis thaliana serine/arginine-rich protein with derived molecular mass of approximately 30 kD). It was >4.5 kb long, had 1805 bp of promoter region, but ended 12 bp upstream of the stop codon. A corresponding cDNA was obtained from an expressed sequence tag (EST) cDNA library of A. thaliana ecotype Columbia and sequenced. The mRNA corresponding to this cDNA was designated mRNA1 of atSRp30. Primers derived from the 3′ untranslated region of this cDNA were used to obtain the missing sequences of the genomic clone by PCR amplification on purified genomic DNA. The DNA fragment contained an additional intron, and the rest was identical to the corresponding sequence of the cDNA1. The sequence of Gat SRp30 and the derived protein sequence are shown in FIG. 1A.

To facilitate comparison of features of at SRp30 and of the closely related Arabidopsis atSRp 34/SR1 protein (Lazar et al. 1995), the genomic sequence of this protein was determined using PCR. Gat SRp 34 has a very similar gene structure to that of Gat SRp30 , except for an intron present in the 5′ untranslated region of the former. The fragment of Gat SRp34 comprising the part from exon 11 to the stop codon is shown in FIG. 1C, where the alternative splicing events involving intron 11 are also indicated.

### Comparison of at SRp30 to other SR Proteins

The derived protein sequences of at SRp30 and atSRp34/SR1 are very homologous (80.7% similarity and 67.1% identity) to each other, and both show very high similarity (75.3 and 77.8%, respectively) and identity (58.1 and 59.4%, respectively) to human SF2/ASF (FIG. 2). As at SRp30 and atSRp34/SR1 are less homologous to other animal or plant SR proteins identified to date, both proteins can be considered true orthologs of human SF2/ASF. Within their amino-terminal portions, all three proteins contain two RRMs with their conserved RNP-2 and RNP-1 submotifs, whereby the second RRM is atypical and contains the invariant SWQDLKD signature characteristic of SF2/ASF-like SR proteins (Birney et al. 1993). However, unlike atSRp34/SR1 and SF2/ASF, the RRMs of at SRp30 are not separated by a glycine-rich ‘hinge’ region but rather by a serine-rich sequence. The RS domain of at SRp30 is shorter than the one of atSRp34 because of an extension at the 3′ end of atSRp34 which includes a previously described positively charged proline/serine/lysine-rich (PSK) domain of unknown function (Lazar et al. 1995). If this unique 3′ extension is not taken into account, atSRp34/SR1 is slightly more homologous to human SF2/ASF, mainly because of their common G-rich hinge region. Taken together, these analyses suggest that in contrast to mammals, for which only one SF2/ASF protein has been described to date, two SF2/ASF homologs exist in Arabidopsis.

### RNA Distribution and Alternative Splicing Forms of at SRp30 and atSRp34/SR1

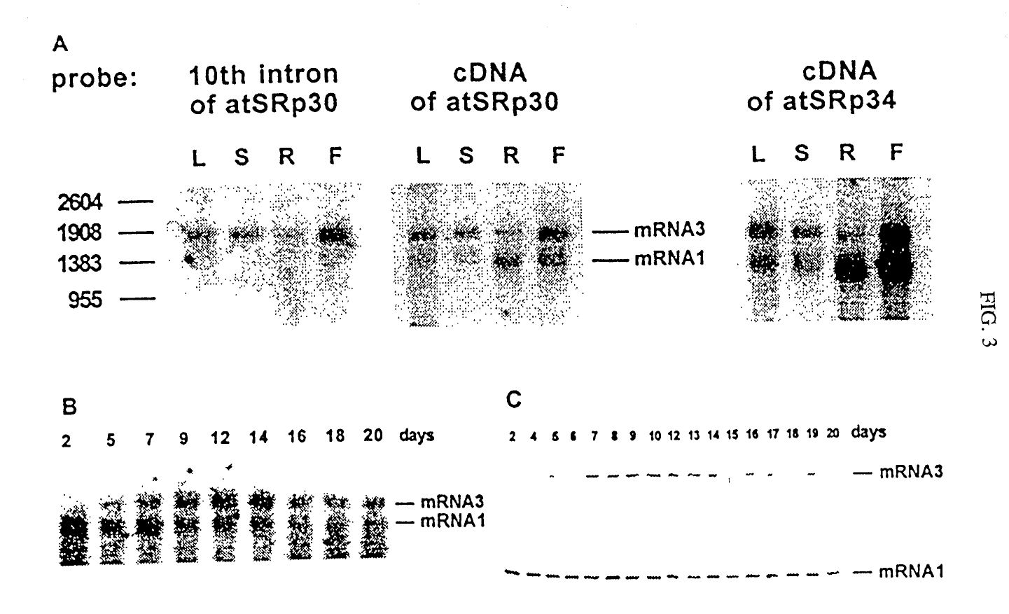

RNA blots of poly (A)⁺ mRNA from various tissues of wild-type Arabidopsis plants were probed with radioactive labeled atSRp30 or atSRp34/SR1 cDNAs and revealed at least two mRNA species in each case (FIG. 3A). The level of expression of each gene varied considerably in different tissues but in both cases was highest in flowers, followed by roots (FIG. 3A). In addition, the ratio of the two discernible mRNAs, mRNA3 and mRNA1, was different for each gene in the various organs. For at SRp30 , mRNA3 seemed to be more abundant in leaves, stems and flowers, whereas mRNA1 was predominant in roots. In contrast, the ratio of the two main mRNAs of atSRp34/SR1 was approximately 1:1 in leaves and stems, whereas in roots and flowers mRNA1predominated. Reprobing of the RNA blot with the long tenth intron of at SRp30 showed that mRNA3 retained sequences of this intron, indicating that alternative splicing involves this region of at SRp30 pre-mRNA.

The full sequences of the various mRNA isoforms were obtained by RT-PCR cloning from total RNA using primers from the 5′ and 3′ ends of the genes (see Materials and Methods). mRNA1corresponded to the cDNA sequence of at SRp30 , whereas mRNA3 retained part of the ₁₀th intron, because of usage of an alternative 3′ splice site (FIG. 1A, B). The mRNA2 isoform with both alternative 3′ and 5′ splice sites within intron 10 (FIG. 1A, B) was not visible in Northerns of wild-type plants (see FIG. 3A) and was only detected by RT-PCR by increasing the number of cycles (data not shown).

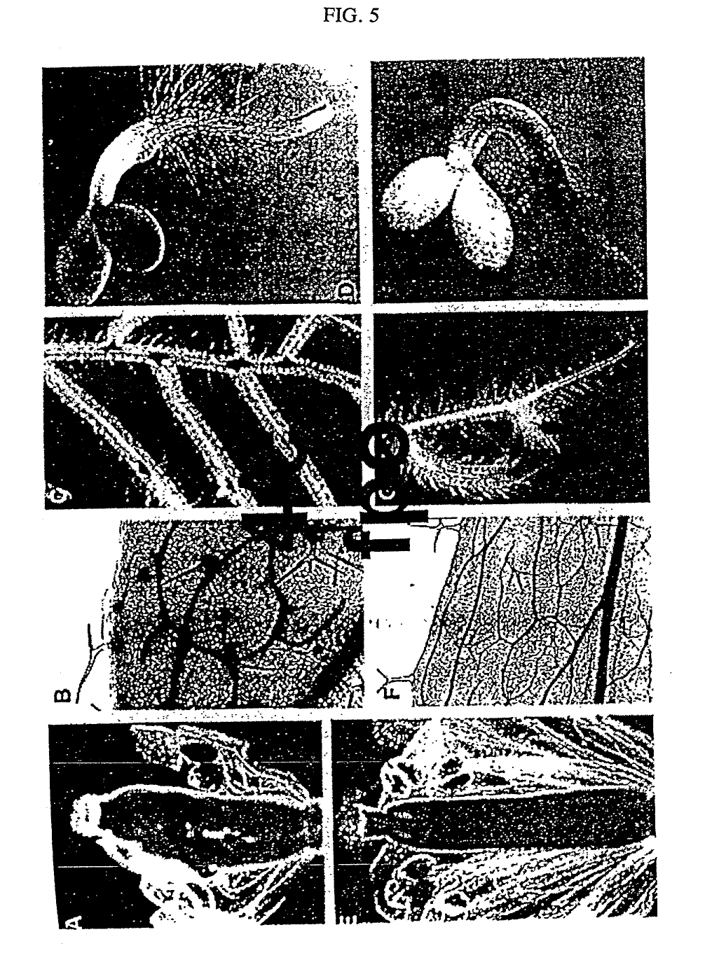

The same approach was used to sequence the mRNA isoforms of atSRp34/SR1 (FIG. 1C). The longest mRNA species, which is also the most abundant in wild-type plants, was named mRNA5 and retained most of intron 11 (which is in the position corresponding to intron 10 of at SRp30 ) except for an internal intron of approximately 80 nucleotides. Two other minor alternative mRNAs could be detected during early plant development, but only by RT-PCR (data not shown). mRNA4 uses an additional alternative 3′ splice site; mRNA2 uses alternative 5′ and 3′ splice sites (FIG. 1C, D); and mRNA3, which is the main alternatively spliced mRNA in plants when at SRp30 is overexpressed from the 35SCaMV promoter (FIG. 5, below) uses one alternative 5′ splice site.

The above alternatively spliced mRNAs of at SRp30 and atSRp34/SR1 all had an in-frame stop codon near the 5′ end of the retained intronic sequences. The hypothetical shorter proteins, designated at SRp30 s and atSRp34s, lack the carboxy-terminal part of the RS domain and instead have other sequences that are shown in bold in FIG. 1A for at SRp30 s and in FIG. 1C for atSRp34s.

To obtain information about changes in the transcriptional and alternative splicing patterns of at SRp30 during plant development, total RNA was isolated from whole plants of different ages and used for RNA blot hybridization and RT-PCR analysis (FIG. 3B, C). Primers derived from exons adjacent to the 10^{th} intron were used for RT-PCR. The results from both methods were in good agreement and showed that the expression of mRNA1is highest in younger plants and starts to decline around day 12, whereas the expression of mRNA3 is extremely low in young seedlings, peaks between days 9-14, and declines slowly afterwards. Although it is not known how the ratio of these two transcripts is regulated, these regulatory events might determine the quantity of genuine at SRp30 protein in individual cells.

### Antibody Detection of Arabidopsis SR Proteins Polyclonal antibodies were raised in chickens against purified recombinant atSRp30 and atSRp34/SR1 and used to identify antigens in different ammonium sulfate fractions of Arabidopsis extracts. The extracts were fractionated by sequential precipitation with ammonium sulfate and magnesium chloride, to enrich for SR proteins (Roth et al. 1990). The anti-p30 and anti-p34 antibodies recognized proteins in the 60%-90% ammonium sulfate part. The immunoreactive proteins in the dialyzed 60%-90% part precipitated quantitatively in the presence of 20 mM magnesium chloride. No crossreaction of plant proteins with the pre-immune immunoglobulin fraction was found (data not shown).

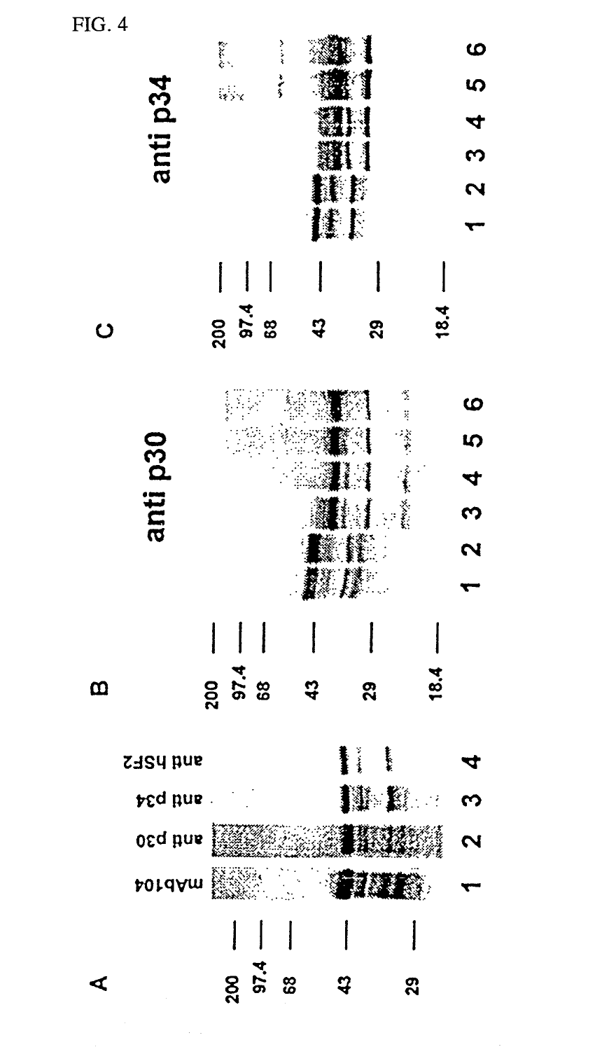

The magnesium precipitate was immunoblotted and probed with four different antibodies. With anti-p30, six protein bands were detected as three doublets, with the 43- to 46-kD doublet showing the highest intensity. The two other doublets migrated with apparent molecular masses of 38-40 and 31-34 kD (FIG. 4A, lane 2). The immunostaining pattern with the monoclonal antibody mAb104 (FIG. 4A, lane 1), which is specific for a serine phosphoepitope common to SR proteins (Roth et al. 1990) was very similar, although the smallest band was more pronounced than with anti-p30 (lane 2). This could be indicative of an additional SR protein of approximately 30 kD, or it may reflect the presence of multiple or stronger phosphoepitopes within p30. No high molecular mass proteins were detected with the mAb104 antibody, although Lazar et al. (1995) have reported immunoreactive proteins up to 120 kD.

The anti-p34 antibodies recognize mainly three proteins of approximately 46-47, 40 and 34 kD. As the same antibody shows minimal cross-reaction with recombinant at SRp30 (data not shown), these bands possibly represent atSRp34/SR1-related proteins and correlate well with published overexpression data in which the protein was immunodetected with a size of 47-48 kD (Lazar et al. 1995).

The 40-kD protein comigrates with an alternatively spliced isoform of atSRp34/SR1 (see below), whereas the nature of the 34-kD polypeptide remains to be determined. Interestingly, the immunoblot with anti-p34 is very similar to that obtained with a monoclonal antibody specific for human SF2/ASF (FIG. 4A, cf. lanes 3 and 4). Therefore, anti-hSF2, which recognizes a discontinuous epitope within RRM1 of hSF2/ASF but does not recognize other human SR proteins (Hanamura et al. 1998) cross-reacts with the same set of Arabidopsis proteins as anti-p34, supporting the notion that hSF2/ASF resembles atSRp34/SR1 in this region more closely than it does at SRp30.

The fact that at SRp30 and atSRp34/SR1 are present in the SR protein preparation and comigrate with specific bands in the mAb104 immunoblot suggests that both proteins are phosphorylated. To test this suggestion, the SR protein preparation was treated with increasing concentrations of alkaline phosphatase and for different times (FIG. 4B, C). Compared to the untreated controls (lanes 1, 2), these treatments resulted in an increase in the mobility of all immunostained bands. Under these conditions, anti-p30 recognized predominantly three proteins (27, 31, and 38 kD), whereas anti-p34 recognized two proteins (31 and 38 kD). As expected, all these proteins were no longer stained with mAb104, except for the 38-kD band, which probably represents a partially dephosphorylated intermediate resistant to further dephosphorylation (data not shown). Interestingly, dephosphorylation led to the appearance of new bands of higher apparent molecular mass, consistent with the observation that unphosphorylated recombinant at SRp30 and atSRp34/SR1 are very insoluble and have a strong tendency to aggregate.

### Transcriptional Activity of SR Protein Promoters in Arabidopsis

It is striking that in contrast to mammals, Arabidopsis possesses two SF2/ASF-like proteins. As revealed by the Northern blot analysis, the genes of both proteins are transcribed in all plant organs. It was therefore of interest to investigate if both genes are generally transcribed in all tissues or specifically in different tissues, or to which extent they have overlapping activities. To this end, the promoters of Gat SRp30 and Gat SRp34, including the 5′ untranslated regions up to the start codon, were fused to the coding region of a reporter β-glucuronidase gene and transferred into A. thalianaplants by Agrobacterium tumefaciens-mediated root transformation. β-Glucuronidase (GUS) activity was never observed in any tested nontransgenic Arabidopsis tissues, as well as in transgenic plants harboring a promoterless GUS gene. Control plants containing a 35 S cauliflower mosaic virus (CaMV) promoter-Gus fusion were easily stained, demonstrating that substrate accessibility was not limiting GUS activity. To exclude an influence of the transgene position on promoter activity, the analyses were carried out with several independent transgenic lines.

Using detailed histochemical analyses, it was found that both genes had distinct but also overlapping patterns of transcriptional activities. Flowers in the postanthesis stage (FIG. 5A, E) showed essentially the same staining patterns for both genes, with the most pronounced expression in the pollen grains and weak staining of sepals and style. Additionally, weak activity was observed in the abscission zone of the flower in at SRp30-GUS transgenic plants (FIG. 5A). The leaves of at SRp30 -GUS plants showed strongest staining in vascular bundles and in the support cells of each trichome (FIG. 5B), whereas the atSRp34-GUS fusion was weakly active only in secondary veins and in the cells around them, but activity was never observed in trichomes or support cells (FIG. 5F). The differences in the GUS staining patterns of these genes were especially evident during the different stages of lateral root development. Both at SRp30 and atSRp34-GUS fusions were active at the very first stages of lateral root initiation, when pericycle cells are stimulated, de-differentiate, and proliferate. Then, at the later stages, when redifferentiation occurs to form the lateral root meristem, at SRp30 -GUS was expressed in the basal enlarged cells of the forming lateral root (FIG. 5C), whereas atSRp34-GUS expression was characteristic of the activated root meristem (FIG. 5G). The staining patterns during early seedling development provided additional evidence for the distinct transcriptional activities of at SRp30 and atSRp34/SR1. Each of the genes was expressed in specific regions along the apical-basal axis of the seedling. In at SRp30 -GUS seedlings, expression was restricted to the cotyledons (FIG. 5D), whereas in atSRp34-GUS seedlings, expression was observed in hypocotyl and in root (FIG. 5H), suggesting that these genes can be transcriptionally active in regions with different patterns of cell division. This suggestion was confirmed by differential expression of at SRp30 and atSRp34/SR1 during lateral root formation, as well as in trichome support cells.

### Overexpression of at SRp30 in Transgenic Plants

In Drosophila, ectopic overexpression of SRp55/B52 resulted in various developmental abnormalities, although the identity of affected transcripts remains unknown (Kraus and Lis 1994). It was therefore of interest to study the effect of overexpression of at SRp30 on plant development and on the alternative splicing patterns of individual plant transcripts. The constructs used contained either a complete gene (pG30) or a cDNA (pC30) encoding at SRp30 under the control of the strong constitutive promoter of the 35S RNA from CaMV. The 35S CaMV promoter is strong and constitutive in all plant tissues studied, which was confirmed in control experiments using GUS as a reporter gene. To control for the conditions of the regeneration and transformation procedure in the RNA analysis of transformants, negative controls were either transformed with a 35S RNA promoter-GUS control (pBI121) or were transformants with the same construct, but which for unknown reasons did not show overexpression.

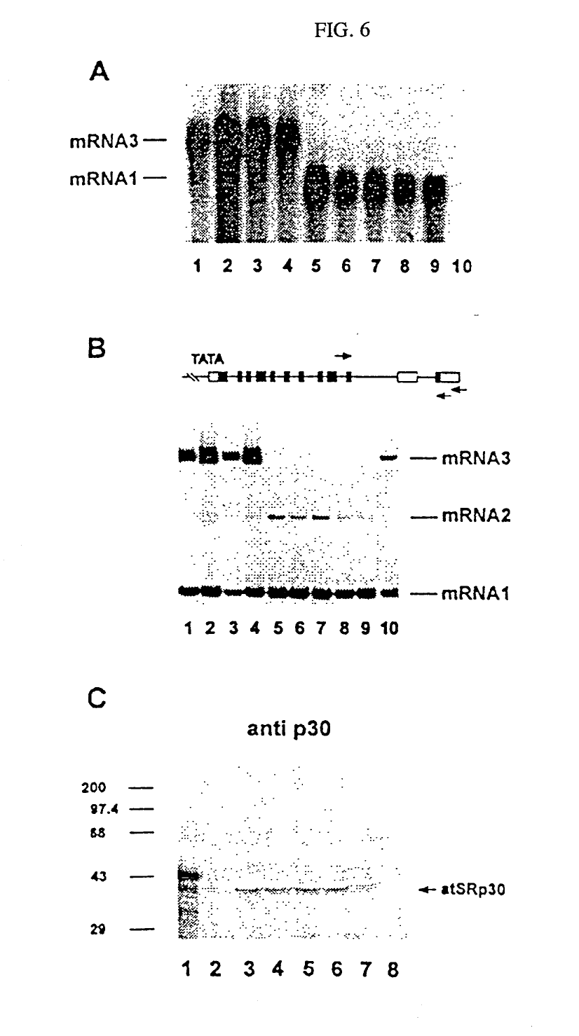

Both fusion constructs, pG30 and pC30, were used for Agrobacterium-mediated transformation of Arabidopsis roots. Forty independent transgenic lines were regenerated for each construct; eight of the lines transformed with pG30 and twelve transformed with pC30 were used in further work. Some of these transgenic lines were used for RNA blot and RT-PCR analyses as shown in FIG. 6A and B. Surprisingly, all pG30 transformants (FIG. 6A, B, lanes 1-4) expressed mainly mRNA3, which possesses an alternative 3′ splice site within the long intron (FIG. 1B), but nevertheless, mRNA1 was still more abundant in these plants than in the control plant (FIG. 6A, lane 10), as judged by the RNA blot analysis (four times more product was loaded in lane 10 in FIG. 6B). Although mRNA1levels in both types of transformants are quite different (FIG. 6A), the RT-PCR shows similar yields (FIG. 6B) because a high number of cycles was employed to detect all alternatively spliced products. As expected, all transformants containing pC30 overexpressed only mRNA1(FIG. 6A, lanes 5-9). In both types of transformants, overexpressed mRNAs were >100-fold more abundant than in wild-type plants. Interestingly, when pC30 transformants were analyzed by RT-PCR, only mRNA2, which uses cryptic 3′ and 5′ splice sites in intron 10 (FIGS. 6B and 1B), was present, whereas a small amount of mRNA3 was visible in wild-type plants. This result shows that an excess of at SRp30 changes 5′-splice-site selection of the endogenous at SRp30 pre-mRNA.

In light of the above results, it was of interest to measure overexpression of at SRp30 at the protein level. Total soluble protein extracts of transgenic plants were analyzed by Western blotting using anti-p30 for immunodetection (FIG. 6C) and compared to a control plant (lane 8, a transformed plant with no overexpression of atSRp30) and to an SR protein preparation from an Arabidopsis suspension culture (lane 1). The SR protein preparation had the characteristic pattern for anti-p30 (cf. FIG. 6C, lane 1 and FIG. 4A, lane 2), whereas no specific proteins were immunostained in the total protein extract of the control plant (FIG. 6C, lane 8), because of the low abundance of at SRp30 in plants. In contrast, a specific protein band was visible in the pC30 transformants (lanes 3-7), comigrating with a protein band in the SR protein preparation (lane 1). In contrast, in the pG30 transformant (lane 2), only a faint protein band was detected. These results correlate well with the RNA expression pattern of mRNA1in both types of transformants (FIG. 6A). It is interesting that overexpression of at SRp30 yielded an immunostained protein of 38 kD and not a band comigrating with the more abundant 43- to 46-kD doublet seen in the SR protein preparation (FIG. 6C, lane 1). One likely explanation is that overexpressed at SRp30 is only partially phosphorylated, which may be related to the observation that even exhaustive dephosphorylation results in a 38-kD at SRp30 protein that retains the mAb104 phosphoepitope (FIG. 4B, lanes 3-6). However, no shorter protein product (at SRp30 s) of the highly abundant mRNA3 in pG30 transformants could be detected with any antibody used (see also FIG. 6C, lane 2).

### Phenotypic Changes in Plants Overexpressing at SRp30

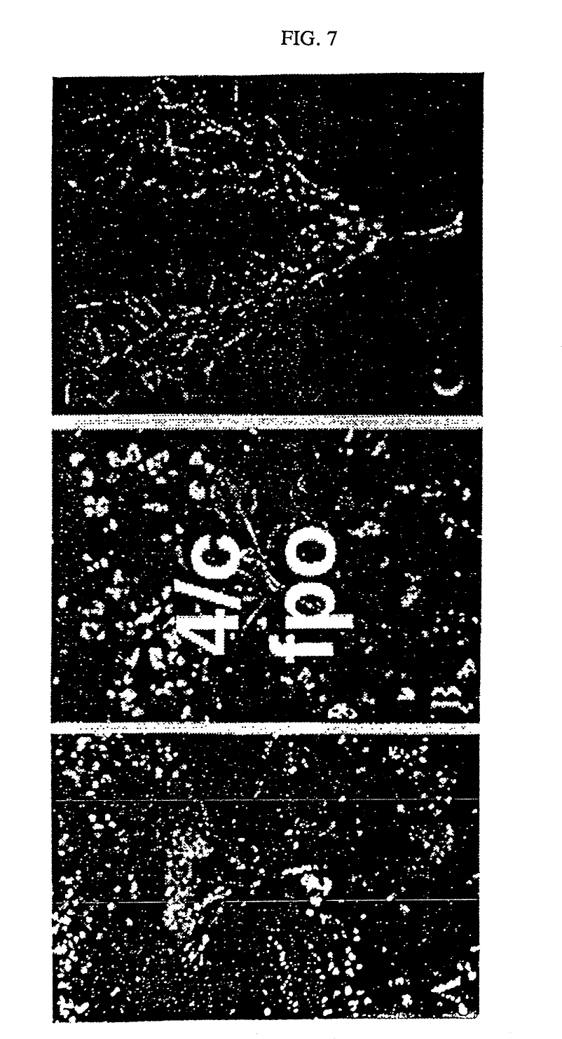

Overexpression of at SRp30 resulted in strong phenotypes with pleiotropic changes both in morphology and development of the transgenic plants. No significant differences were observed between plants transformed with pG30 and pC30 constructs, although the levels of at SRp30 protein were different (FIG. 6C, cf. lane 2 and lanes 3-7). The observations were reproducible in T₂ and subsequent generations of independent transgenic lines, and cosegrated with antibiotic resistance. In transgenic plants, the transition from vegetative to flowering stage was delayed greatly under short day conditions. The time from germination to the formation of the first mature silique was 65-78 days in overexpressing plants, compared to 42-47 days in control plants grown under the same conditions. In addition, adult plants showed reduced apical dominance, resulting in a ‘bushy’ phenotype with an increased number of secondary inflorescences and a shortened primary inflorescence (FIG. 7C). Growing of the transgenic plants under long-day conditions resulted in partial reversion to a normal phenotype. But despite of the partial recovery of apical dominance, the transition to flowering and the time to senescence remained delayed. Under long-day conditions, the majority of plants from overexpressing lines started to flower between 35 and 39 days after germination, whereas control plants flowered between days 25 and 28. The initial inflorescences were very short and did not exceed 0.5-1 cm when the first flower opened. The size of flowers in transgenic plants was approximately 30% larger than in control plants (4 and 3 mm, respectively). Transgenic plants also had larger rosette leaves (FIG. 7A, cf. to wild type in B) with trichomes having predominantly four to five branches, compared to wild-type leaves, which had mainly three-branched trichomes. In strong overexpressing lines, the primary inflorescence produced numerous secondary branches with a vegetative rosette-like appearance. These branches developed from 7-15 vegetative-like leaves and finally formed inflorescences. Under long-day conditions, the rosette leaf numbers determined at the time of flowering were somewhat lower in transgenic lines compared to the wild type (an average of 12 and 16 leaves, respectively). Independent transgenic lines overexpressing at SRp30 displayed the described characteristics to various extents, with the main impact on the time for transition from the vegetative to the reproductive phase.

### Overexpressed at SRp30 Modulates Alternative Splicing In Vivo

The influence of overexpression of at SRp30 on its own alternative splicing pattern and the high homology of at SRp30 to human alternative splicing factor SF2/ASF suggested that this protein could be a modulator of plant splicing. This assumption cannot be tested in vitro, as no plant splicing extracts are available. Therefore, total RNA preparations from pG30 transformants (lower level of at SRp30 overexpression) and pC30 transformants (higher level of at SRp30 overexpression) were used for RT-PCR analysis of several plant introns that are known to be processed alternatively under wild-type conditions.

Among several genes tested, such as FCA (Macknight et al. 1997), rubisco activase (Werneke et al. 1989), agamous (Yanofsky et al. 1990), and LSD1 (Dietrich et al. 1997), no changes in the splicing patterns were observed in plants overexpressing at SRp30. However, pre-mRNA splicing of three plant genes was found to be affected by overexpression, as described below.

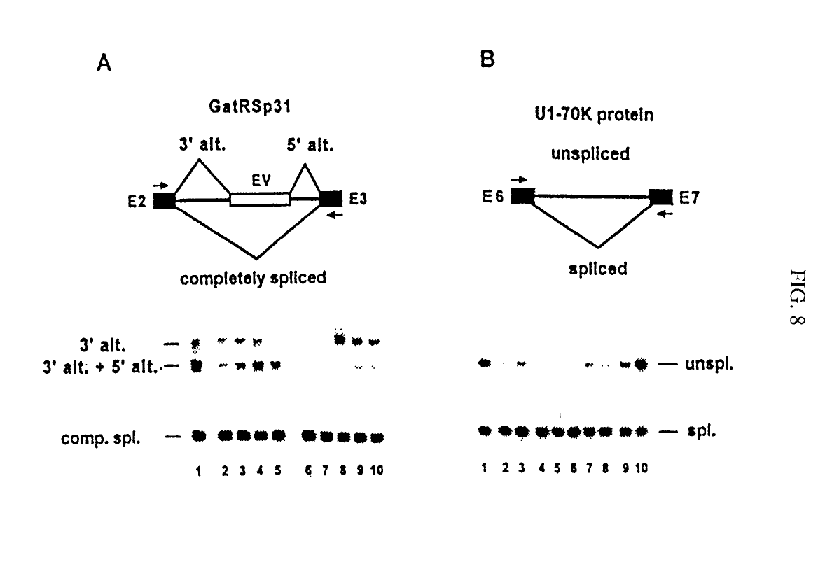

The splicing factor atRSp31 belongs to a novel family of plant RS proteins and possesses an alternatively spliced second intron (792 nucleotides) (Lopato et al. 1996b). In the wild-type plant, either a cryptic 3′ splice site or both alternative 3′ and 5′ splice sites were used in this intron (FIG. 8A), whereas the latter form prevailed in control lines (lane 1) and pG30 transformed lines (lanes 2-5). In contrast, pC30 lines (lanes 6-10) expressed mainly the alternative 3′ splice site form, although its abundance was variable (in lanes 6 and 7 this form is only visible in the original photograph). These transcripts are probably subject to nonsense-mediated decay and potentially code for the same truncated protein.

Another well-described alternative splicing event occurs in the case of the U1-70K gene of Arabidopsis, in which transcripts retaining intron 6 are more abundant in most tissues (except roots) than the correctly spliced transcript (Golovkin and Reddy 1996). The RT-PCR results from control plants (FIG. 8B, lanes 1, 9, 10) confirm this observation. However, in both at SRp30 transformants, either the lower expressing pG30 lines (lanes 2 and 3) or the higher expressing pC30 lines (lanes 4-8), most of the transcripts were spliced correctly and only a low level of intron retention was observed. This regulatory event could potentially influence the level of active protein.

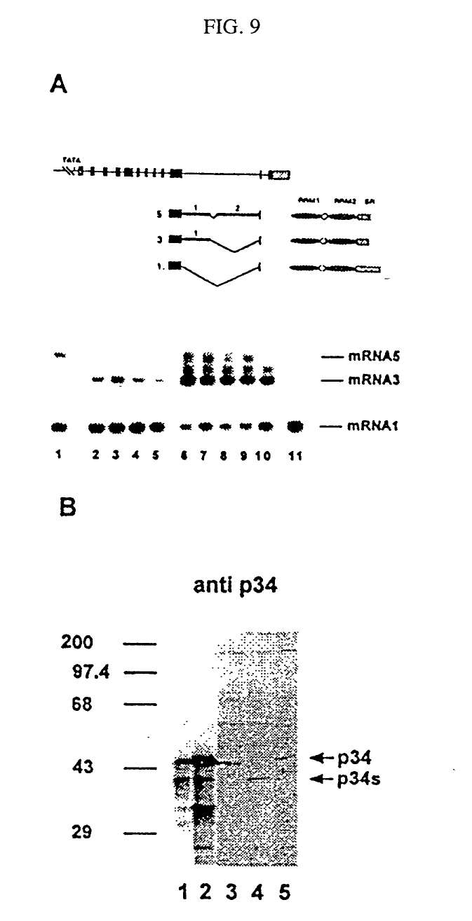

The most interesting case of alternative splicing modulation was found in the case of the long intron (intron 11) of Gat SRp34, which is a close homolog of Gat SRp30 (FIG. 9). In wild-type plants (FIG. 9, lane 1), part of this intron is alternatively spliced to generate mRNA5. However, in pG30 transformants (lanes 2-5), mRNA3 was the main alternative spliced form, whereas the level of mRNA1 appeared unchanged. Surprisingly, when at SRp30 was highly overexpressed, the alternatively spliced mRNA3 became the main transcript, whereas the amount of mRNA1 was considerably decreased. Assuming that shorter PCR products are probably overrepresented, the reduction of mRNA1 may be even more drastic. This assumption was confirmed by immunoblot analysis of total protein extracts: atSRp34/SR1 was deleted in control plants (FIG. 9B, lane 3) and in a pG30 transformant (lane 5), but not in a pC30 transformant (lane 4). However, a smaller specific protein band (atSRp34s, 38 kD) became visible in this case, indicating that the protein product of mRNA3 predominates in this transformant. These results clearly show that overexpression of at SRp30 strongly influences gene expression of its close homolog, atSRp34/SR1.

### DISCUSSION

### at SRp30 is a Member of the SR Protein Family

In addition to their characteristic domains, which include one or two RRMs and a carboxy-terminal RS domain with multiple SR dipeptides, SR proteins possess phosphoepitope(s) recognized by mAb104, and they are insoluble in the presence of millimolar concentrations of magnesium salts. The antibody to at SRp30 recognized a complex pattern of bands in the Mg precipitate, whereas in total protein fractions no specific band could be detected. The failure to detect at SRp30 in crude lysates reflects the low abundance of the protein and/or the limited sensitivity of the antibody. However, when at SRp30 was overexpressed, the antibody detected a specific protein with an apparent mobility of 40 kD. In contrast, atSRp34/SR could be detected by anti-p34 in total protein fractions. The immunoblot data confirm that at SRp30 is a true SR protein, as it is present in SR protein preparations and shows corresponding immunostained bands with mAb104. However, as complete dephosphorylation could not be achieved, it is not known how many of these proteins represent modified forms of at SRp30 or closely related proteins.

### Is the expression of at SRp30 autoregulating?

When at SRp30 pre-mRNA was overexpressed, mRNA3, which is generated from use of an alternative 3′ splice site in the tenth intron, was the main transcript detected, whereas the level of mRNA1 was enhanced only moderately (FIG. 6). There are several possible explanations for this phenomenon. First, there may be a limiting splicing factor that is titrated out because of overexpression of the at SRp30 gene. Such a factor would have to be specific for the tenth intron, as all the other introns in at SRp30 are spliced properly. Second, mRNA 3 and mRNA1 may be synthesized for the most part in different cells, via cell-type-specific alternative splicing. As the constitutive 35S CaMV promoter used in the overexpression experiments is active in most tissues the large amount of mRNA3 may reflect inappropriate expression in cell types in which a factor required for correct processing of the tenth intron is absent. To clarify this issue, experiments aimed at determining the cells in which the alternative splicing event takes place will be necessary.

The fact that the ratio of mRNA3 to mRNA1 is constant in all transformants overexpressing Gat SRp30 (FIG. 6A) is consistent with a potential autoregulatory mechanism involving at SRp30. Overproduced at SRp30 protein may stimulate the alternative splicing event, thus down-regulating its own expression, as has been shown to be the case for several animal splicing factors, such as Sxl (Bell et al. 1991), tra (Mattox and Baker 1991), and SWAP (Zachar et al. 1987) from Drosophila, as well as mouse SRp20 (Jumaa and Nielsen 1997). In agreement with this model, it was not possible to detect a protein product from mRNA3 (at SRp30 s). Evidence was obtained for an influence of at SRp30 on alternative splicing of transcripts from the endogenous gene in plants overexpressing the at SRp30 cDNA (FIG. 6B, lanes 5-9), resulting in preferential use of an alternative 5′ splice site. Overexpression of at SRp30 might have a different effect on the large amount of exogenous pre-mRNA present in cells where this protein is not expressed normally resulting in preferential use of an alternative 3′ splice site (FIG. 6A, B, lanes 1-4). Unfortunately, this hypothesis cannot be tested in vitro because no plant splicing extracts are available. Future experiments should address whether at SRp30 can indeed autoregulate its own production.

### at SRp30 Overexpression Influences Splice-site Choice in Several Plant Pre-mRNAs and Induces Changes in Plant Development

Many in vivo and in vitro experiments have shown that human SF2/ASF is involved in the selection of 5′ splice sites by binding cooperatively to this site (Zuo and Manley 1994) and to U1snRNP (Kohtz et al. 1994; Jamison et al. 1995; Zahler and Roth 1995). Furthermore, like other SR proteins, SF2/ASF influences splice-site selection in part by binding to enhancer sequences that are present frequently in intronic or exonic regions. Upon binding to those elements, the protein activates splicing by recruiting the splicing mechanism to an adjacent splice site through protein-protein interactions (Wu and Maniatis 1993; Amrein et al. 1994; Zuo and Manley 1994). Arabidopsis at SRp30 was a good candidate for being a splicing modulator, given its similarity to human SF2/ASF and its peculiar expression pattern. The ability to stably overexpress at SRp30 in whole plants that remained viable made it possible to determine, for the first time, the effects of increased SR protein levels on alternative splicing of specific endogenous transcripts. Some but not all endogenous transcripts tested were affected. In the case of at SRp30, pre-mRNA itself, and tSRp34/SR1/pre-mRNA, intronic alternative 5′ splice sites were activated, whereas in atRSp31 and U1 70K pre-mRNAs, the usage of the normal splice sites was generally enhanced but variable. There is no explanation for this variability in individual transformants (see FIG. 8), as the at SRp30 protein levels in several pC30 transformants were similar (FIG. 6C). The most pronounced effect of at SRp30 overexpression was on the splicing pattern of the close homolog atSRp34/SR1, in which preferential use of the alternative ₅′ splice site gave rise mostly to a truncated mRNA isoform, atSRp34s, and virtually no atSRp34/SR1 protein. Although there is no information to date concerning the activity of the smaller atSRp34s, these results suggest the normal expression of at SRp30 protein in the same cells that transcribe atSRp34/SR1 is important for the synthesis of authentic atSRp34/SR1 protein. This regulatory loop might explain the different expression patterns of both factors in root tissue and young seedlings. It remains to be determined whether there is a reciprocal effect of atSRp34/SR1 overexpression on alternative splicing of at SRp30 pre-mRNA.

Plants overexpressing at SRp30 constitutively and ubiquitously showed interesting changes in morphology, as well as in several aspects of development. Although some of the details will have to be investigated more thoroughly, many of the differences can be explained by changes in phase transition and in the definition of cell fates. In this work it has been demonstrated that at SRp30 can change the expression of other genes drastically by influencing their splicing patterns. As the expression pattern of at SRp30 is normally very tissue specific, some of the observed effects of overexpression might simply be caused by its expression in inappropriate tissues, where it influences the expression of genes that are not the natural targets of this splicing factor. In addition, the observed decrease in the expression of the splicing factor atSRp34 upon overexpressing at SRp30 may in turn affect the expression of other genes, leading to the observed phenotypic changes. On the other hand, the changes in trichome development resulting in additional branching may reflect overexpression of at SRp30 in the supporting cell of the trichomes, which is a normal site of at SRp30 expression. Therefore, at SRp30 may be a determinant of trichome development. Finding the natural targets of regulation by at SRp30 remains a critical goal to explain the observed changes in phenotype, as well as to promote the understanding of developmental pathways in plants.

### Two SF2/ASF-like Factors in Arabidopsis

Immunoblot analysis with mAb104 has suggested previously that the complexity of the SR protein family is higher in animals than in plants (Lopato et al. 1996a), as several higher molecular mass proteins have been identified in SR preparations from mammals but not from those in plants. The existence of two SF2/ASF-like factors in Arabidopsis may compensate for the absence of orthologs of other members of the SR protein family. The similar gene structure of at SRp30 and atSRp34/SR1 is indicative of an ancestral gene duplication event. Interestingly, the penultimate long intron is conserved in both genes and is involved in alternative splicing events. Judging from Northern blot analysis of RNAs from different plant organs, the ratio of the various SR protein mRNAs is quite variable and is probably reflected in the abundance of the resulting proteins. In both genes, the alternative splicing event results in predicted proteins with a truncated RS region and a few new additional carboxy-terminal amino acids. Whereas a shorter protein of atSRp34/SR1 could be detected clearly in plants overexpressing at SRp30, at SRp30s was never observed in these plants, although the corresponding alternatively spliced mRNA is abundant. Expression of at SRp30s cDNA in Escherichia coli yielded stable protein (data not shown). Therefore, either mRNA3 is not translated efficiently, or it is degraded through a premature nonsense codon-mediated decay pathway, or at SRp30s is preferentially recognized by one of the plant protein-degradation systems.

Comparative analysis of at SRp30 with all known plant and animal SR proteins revealed that it is a paralog of atSRp34/SR1 and that both Arabidopsis proteins are closest to human SF2/ASF. The main structural difference is that at SRp30 lacks a G-rich region between the two RRMs, which may influence the flexibility and perhaps the specificity of the RNA-binding region. The notion that the two proteins might have distinct activities is strengthened by the observation that in many cases their expression patterns are quite different, as shown in FIG. 5. In general, at SRp30 expression is more confined to specialized cell types and tissues, like trichomes, cotyledons, or lateral root primordia, suggesting a special role for this protein in the initiation of organ formation, whereas atSRp34/SR1 is more strongly expressed in meristematic tissue. Furthermore, the present sequence and immunological data indicate that atSRp34/SR1 is more similar to hSF2/ASF than is at SRp30. Taken together, these data suggest that atSRp34/SR1 is a more general splicing factor, similar to hSF2/ASF, whereas at SRp30 might have more specialized functions, perhaps acting as a regulatory splicing factor that modulates alternative splicing and gene expression in specific cell types.

### BIBLIOGRAPHY

Allain, F. H. T. and P. W. A. Howe. 1997. Structural basis of the RNA-binding specificity of human U1A protein. EMBO J. 16: 5764-5774.

Amrein, H., M. L. Hedley, and T. Maniatis. 1994. The role of specific protein-RNA and protein-protein interactions in positive and negative control of pre-mRNA splicing by Transformer 2. Cell 76: 735-746.

Barta, A., K. Sommergruber, D. Thompson, K. Hartmuth, M. A. Matzke, and A. J. M. Matzke. 1986. The expression of a no-paline synthase-human growth hormone chimaeric gene in transformed tobacco and sunflower callus tissue. Plant Mol. Biol. 6:347-357.

Bell, L. R., J. I. Horabin, P. Schedl, and T. W. Cline. 1991. Positive autoregulation of sex-lethal by alternative splicing maintains the female determined state in Drosophila. Cell 65: 229-239.

Barney, E., S. Kumar, and A. R. Krainer. 1993. Analysis of the RNA-recognition motif and RS and RGG domains: Conservation in metazoan pre-mRNA splicing factors. Nucleic Acids Res. 21: 5803-5816.

Breitbart, R. E., A. Andreadis, and B. Nadal Ginard. 1987. Alternative splicing: A ubiquitous mechanism for the generation of multiple protein isoforms from single genes. Annu. Rev. Biochem. 56: 467-495.

Brown, J. W. S. and C. G. Simpson. 1998. Splice site selection in plant pre-mRNA splicing. Ann. Rev. Plant Physiol. Plant Mol. Biol. 49: 77-95.

Cáceres, J. F. and A. R. Krainer. 1993. Functional analysis of pre-mRNA splicing factor SF2/ASF structural domains. EMBO J. 12: 4715-4726.

- - - . 1997. Mammalian pre-mRNA splicing factors. In Eucaryotic mRNA processing (ed. A. R. Krainer), S. 174-212. IRL Press, Oxford, U.K.

Cáceres. J. F., S. Stamm, D. M. Helfman, and A. R. Krainer. 1994. Regulation of alternative splicing in vivo by overexpression of antagonistic splicing factors. Science 265: 1706-1709.

Cáceres, J. F., T. Misteli, G. R. Screaton, D. L. Spector, and A. R. Krainer. 1997. Role of the modular domains of SR proteins in subnuclear localization and alternative splicing specificity. J. Cell Biol. 138: 225-238.

Cáceres, J. F., G. R. Screaton, and A. R. Krainer. 1998. A specific subset of SR proteins shuttles continuously between the nucleus and the cytoplasm. Genes & Dev. 12: 55-66.

Cavaloc, Y., M. Popielarz, J. P. Fuchs, R. Gattoni, and J. Stevenin. 1994. Characterization and cloning of the human splicing factor 9G8: A novel 35 kDa factor of the serine/arginine protein family. EMBO J. 13: 2639-2649.

Chabot, B. 1996. Directing alternative splicing: Cast and scenarios. Trends Genet. 12: 472-478.

Diamond, R. H., K. Du, V. M. Lee., K. L. Mohn. B. A. Haber, D. S. Tewari, and R. Taub. 1993. Novel delayed-early and highly insulin-induced growth response genes. Identification of HRS, a potential regulator of alternative pre-mRNA splicing. J. Biol Chem. 268: 15185-15192.

Dietrich, R. A., M. H. Richberg, R. Schmid, C. Dean, and J. L. Dangl. 1997. A novel zinc finger protein is encoded by the Arabidopsis LSD1 gene and functions as a negative regulator of plant cell death. Cell 88: 685-694.

Ditta, G., S. Stanfield, D. Corbin, and D. R. Helinski. 1980. Broad host range DNA cloning system for gram-negative bacteria: Construction of a gene bank of Rhizobium meliloti. Proc. Natl. Acad. Sci. 77: 7347-7351.

Filipowicz, W., M. Gniadkowski, U. Klahre, and H.-X. Liu. 1995. Pre-mRNA splicing in plants. In Pre-mRNA processing (ed. A. Lamond), S. 66-77. R. G. Landes Publishers, Georgetown, Tex.

Fu, X. D. 1993. Specific commitment of different pre-mRNAs to splicing by single SR proteins. Nature 365: 82-85.

- - -. 1995. The superfamily of arginine/serine-rich splicing factors. RNA 1: 663-680.

Fu, X. D. and T. Maniatis. 1992. Isolation of a complementary DNA that encodes the mammalian splicing factor SC35. Science 256: 535-538.

Gamborg, O. L., R. A. Miller, and K. Ojima. 1968. Nutrient requirements of suspension cultures of soybean root cells. Exp. Cell. Res. 50: 151-158.

Ge, H. and J. L. Manley. 1990. A protein factor, ASF, controls cell-specific alternative splicing of SV40 early pre-mRNA in vitro. Cell 62: 25-34.

Ge, H., P. Zuo, and J. L. Manley. 1991. Primary structure of the human splicing factor ASF reveals similarities with Drosophila regulators. Cell 66: 373-382.

Golovkin, M. and A. S. Reddy. 1996. Structure and expression of a plant U1 snRNP 70K gene: Alternative splicing of U1 sn-RNP 70K pre-mRNAs produces two different transcripts. Plant Cell 8: 1421-1435.

Hanamura, A., J. F. Cáceres, A. Mayeda, B. R. Franza, Jr., and A. R. Krainer. 1998. Regulated tissue-specific expression of antagonistic pre-mRNA splicing factors. RNA 4: 430-444.

Hedley, M. L., H. Amrein, and T. Maniatis. 1995. An amino acid sequence motif sufficient for subnuclear localization of an arginine/serine-rich splicing factor. Proc. Natl. Acad. Sci. 92: 11524-11528.

Hoekema, A., P. R. Hirsch, P. J. J. Hooykaas, and R. A. Schilperoort. 1983. A binary plant vector strategy based on separation of vir and T-region of the A. tumefaciens Ti-plasmid. Nature 303: 179-180.

Huang, H. and H. Ma. 1992. An improved procedure for transforming Arabidopsis thaliana root explant. Plant Mol. Biol. Rep. 10: 372-383.

Jamison, S. F., Z. Pasman, J. Wang, C. Will, R. Luhrmann, J. L. Manley, and M. A. Garcia-Blanco. 1995. U1 snRNP-ASF/SF2 interaction and 5′ splice site recognition: Characterization of required elements. Nucleic Acids Res. 23: 3260-3267.

Jefferson, R. A. 1987. Assaying chimeric genes in plants: The GUS gene fusion system. Plant Mol. Biol. Rep. 5: 387-405.

Jumaa, H. and P. J. Nielsen. 1997. The splicing factor SRp20 modifies splicing of its own mRNA and ASF/SF2 antagonizes this regulation. EMBO J. 16: 5077-5085.

Jumaa, H., J. L. Guenet, and P. J. Nielsen. 1997. Regulated expression and RNA processing of transcripts from the SRp20 splicing factor gene during the cell cycle. Mol. Cell Biol. 17: 3116-3124.

Kim, Y. J., P. Zuo, J. L. Manley, and B. S. Baker. 1992. The Drosophila RNA-binding protein RBP1 is localized to transcriptionally active sites of chromosomes and shows a functional similarity to human splicing factor ASF/SF2. Genes & Dev. 6: 2569-2579.

Kohtz, J. D., S. F. Jamison, C. L. Will, P. Zuo, R. Luhrmann, M. A. Garcia Blanco, and J. L. Manley. 1994. Protein-protein interactions and 51-splice-site recognition in mammalian mRNA precursors. Nature 368: 119-124.

Krainer, A. R., G. C. Conway, and D. Kozak. 1990a. The essential pre-mRNA splicing factor SF2 influences 5′ splice site selection by activating proximal sites. Cell 62: 35-42.

- - - . 1990b. Purification and characterization of pre-mRNA splicing factor SF2 from HeLa cells. Genes & Dev. 4: 1158-1171.

Kraus, M. E. and J. T. Lis. 1994. The concentration of B52, an essential splicing factor and regulator of splice site choice in vitro, is critical for Drosophila development. Mol. Cell Biol. 14: 5360-5370.

Lazar, G., T. Schaal, T. Maniatis, and H. M. Goodman. 1995. Identification of a plant serine-arginine-rich protein similar to the mammalian splicing factor SF2/ASF. Proc. Natl. Acad. Sci. 92: 7672-7676.

Li, H. and P. M. Bingham. 1991. Arginine/serine-rich domains of the su(wa) and tra RNA processing regulators target proteins to a subnuclear compartment implicated in splicing. Cell 67: 335-342.

Lopato, S., A. Mayeda, A. Krainer, and A. Barta. 1996a. Pre-mRNA splicing in plants: Characterization of SR splicing factors. Proc. Natl. Acad. Sci. 93: 3074-3079.

Lopato, S., E. Waigmann, and A. Barta. 1996b. Characterization of a novel arginine/serine-rich splicing factor in Arabidopsis. Plant Cell 8: 2255-2264.

Lopato, S., R. Gattoni, G. Fabini, J. Stevenin, and A. Barta. 1999. A novel family of plant splicing factors with a Zn knuckle motif: Examination of RNA binding and splicing activities. Plant Mol. Biol. 39: 761-773.

Lou, H., R. F. Gagel, and S. M. Berget. 1996. An intron enhancer recognized by splicing factors activates polyadenylation. Genes & Dev. 10: 208-219.

Luehrsen, K. R., S. Taha, and V. Walbot. 1994. Nuclear pre-mRNA processing in higher plants. Prog. Nucleic Acid Res. Mol. Biol. 47: 149-193.

Macknight, R., I. Bancroft, T. Page, C. Lister, R. Schmidt, K. Love, L. Westphal, G. Murphy, S. Sherson, C. Cobbett, and C. Dean. 1997. FCA, a gene controlling flowering time in Arabidopsis, encodes a protein containing RNA-binding domains. Cell 89: 737-745.

Manley, J. L. and R. Tacke. 1996. SR proteins and splicing control. Genes & Dev. 10: 1569-1579.

Mattox, W. and B. S. Baker. 1991. Autoregulation of the splicing of transcripts from the transformer-2 gene of Drosophila. Genes & Dev. 5: 786-796.

Mayeda, A. and A. R. Krainer. 1992. Regulation of alternative pre-mRNA splicing by hnRNP Al and splicing factor SF2. Cell 68: 365-375.

Mayeda, A., A. M. Zahler, A. R. Krainer, and M. B. Roth. 1992. Two members of a conserved family of nuclear phosphoproteins are involved in pre-mRNA splicing. Proc. Natl. Acad. Sci. 89: 1301-1304.

Mayeda, A., S. H. Munroe, J. F. Cáceres, and A. R. Krainer. 1994. Function of conserved domains of hnRNP Al and other hnRNP A/B proteins. EMBO J. 13: 5483-5495.

Morrison, M., K. S. Harris, and M. B. Roth. 1997. smg mutants affect the expression of alternatively spliced Sr protein mRNAs in Caenorhabditis elegans. Proc. Natl. Acad. Sci. 94: 9782-9785.

Murashige, T. and F. Skoog. 1962. A revised medium for rapid growth and bioassays with tobacco tissue culture. Plant Physiol. 15: 473-497.

Murray, M. G. and W. F. Thompson. 1980. Rapid isolation of high molecular weight plant DNA. Nucleic Acids Res. 8: 4321-4325.

Ramchatesingh, J., A. M. Zahler, K. M. Neugebauer, M. B. Roth, and T. A. Cooper. 1995. A subset of SR proteins activates splicing of the cardiac troponin T alternative exon by direct interactions with an exonic enhancer. Mol. Cell Biol. 15: 4898-4907.

Roth, M. B., C. Murphy, and J. G. Gall. 1990. A monoclonal antibody that recognizes a phosphorylated epitope stains lampbrush chromosome loops and small granules in the amphibian germinal vesicle. J. Cell Biol. 111: 2217-2223.

Roth, M. B., A. M. Zahler, and J. A. Stolk. 1991. A conserved family of nuclear phosphoproteins localized to sites of polymerase II transcription. J. Cell Biol. 115: 587-596.

Screaton, G. R., J. F. Cáceres, A. Mayeda, M. V. Bell, M. Plebanski, D. G. Jackson, J. I. Bell., and A. R. Krainer. 1995. Identification and characterization of three members of the human SR family of pre-mRNA splicing factors. EMBO J. 14: 4336-4349.

Solymosy, F. and T. Pollak. 1993. Uridylate-rich small nuclear RNAs (UsnRNAs), their genes and pseudogenes, and Usn-RNPs in plants: Structure and function. A comparative approach. Crit. Rev. Plant Sci. 12: 275-369.

Staknis, D. and R. Reed. 1994. SR proteins promote the first specific recognition of pre-mRNA and are present together with the U1 small nuclear ribonucleoprotein particle in a general splicing enhancer complex. Mol. Cell Biol. 14: 7670-7682.

Sun, Q., A. Mayeda, R. K. Hampson, A. R. Krainer, and F. M. Rottman. 1993. General splicing factor SF2/ASF promotes alternative splicing by binding to an exonic splicing enhancer. Genes & Dev. 7: 2598-2608.

Tacke, R. and J. L. Manley. 1995. The human splicing factors ASF/SF2 and SC35 possess distinct, functionally significant RNA binding specificities. EMBO J. 14:3540-3551.

Tacke, R., Y. Chen, and J. L. Manley. 1997. Sequence-specific RNA binding by an SR protein requires RS domain phosphorylation: Creation of an SRp4O-specific splicing enhancer. BioEssays 19: 189-192.

Tian, M. and T. Maniatis. 1993. A splicing enhancer complex controls alternative splicing of doublesex pre-mRNA. Cell 74: 105-114.

Valcarcel, J. and M. R. Green. 1996. The SR protein family: Pleiotropic functions in pre-mRNA splicing. Trends Biochem. 21: 296-301.

Valvekens, D., M. Van Montague, and M. Van Lijsebettens. 1988. A. tumefaciens-mediated transformation of Arabidopsis thaliana root explants using kanamycin selection. Proc. Natl. Acad. Sci. 85: 5536-5540.

Van Santen, V. L. and R. A. Spritz. 1987. Splicing of plant pre-mRNAs in animal systems and vice versa. Gene 56: 253-265.

Vellard, M., A. Sureau, J. Soret, C. Martinerie, and B. Perbal. 1992. A potential splicing factor is encoded by the opposite strand of the trans-spliced c-myb exon. Proc. Natl. Acad. Sci. 89: 2511-2515.

Wang, J. and J. L. Manley. 1995. Overexpression of the SR proteins ASF/SF2 and SC35 influences alternative splicing in vivo in diverse ways. RNA 1: 335-346.

Wang, R., R. Kobayashi, and J. M. Bishop. 1996. Cellular adherence elicits ligand-independent activation of the Met cell-surface receptor. Proc. Natl. Acad. Sci. 93:8425-8430.

Werneke, J. M., J. M. Chatfield, and W. L. Ogren. 1989. Alternative mRNA splicing generates the two ribulosebiphosphate carboxylase/oxygenase activase polypeptides in spinach and Arabidopsis. Plant Cell 1: 815-825.

Wiebauer, K., J.-J. Herrero, and W. Filipowicz. 1988. Nuclear pre-mRNA processing in plants: Distinct modes of 3′-splice-site selection in plants and animals. Mol. Cell. Biol. 8: 2042-2051.

Wu, J. Y. and T. Maniatis. 1993. Specific interactions between proteins implicated in splice site selection and regulated alternative splicing. Cell 75: 1061-1070.

Xiao, S. H. and J. L. Manley. 1997. Phosphorylation of the ASF/SF2 RS domain affects both protein-protein and protein-RNA inter actions and is necessary for splicing. Proc. Natl. Acad. Sci. 94: 1148-1153.

Yang, X., M. R. Bani, S. J. Lu, S. Rowan, Y. Ben David, and B. Chabot. 1994. The A1 and A1B proteins of heterogeneous nuclear ribonucleoparticles modulate 5′ splice site selection in vivo. Proc. Natl. Acad. Sci. 91: 6924-6928.

Yanofsky, M. F., H. Ma, J. L. Bowman, G. N. Drews, K. A. Feldmann, and E. M. Meyerowitz. 1990. The protein encoded by the Arabidopsis homeotic gene agamous resembles transcription factors. Nature 346: 35-39.

Zachar, Z., D. Garza, T. B. Chou, J. Goland, and P. M. Bingham. 1987. Molecular cloning and genetic analysis of the suppressor-of-white-apricot locus from Drosophila melanogaster. Mol. Cell Biol. 7: 2498-2505.

Zahler, A. M. and M. B. Roth. 1995. Distinct functions of SR proteins in recruitment of U1 small nuclear ribonucleoprotein to alternative 5′ splice sites. Proc.

Natl. Acad. Sci. 92: 2642-2646.

Zahler, A. M., W. S. Lane, J. A. Stolk, and M. B. Roth. 1992. SR proteins: A conserved family of pre-mRNA splicing factors. Genes & Dev. 6: 837-847.

Zahler, A. M., K. M. Neugebauer, W. S. Lane, and M. B. Roth. 1993. Distinct functions of SR proteins in alternative pre-mRNA splicing. Science 260: 219-222.

Zuo, P. and T. Maniatis. 1996. The splicing factor U2AF35 mediates critical protein-protein interactions in constitutive and enhancer-dependent splicing. Genes & Dev. 10: 1356-1368.

Zuo, P. and J. L. Manley. 1993. Functional domains of the human splicing factor ASF/SF2. EMBO J. 12: 4727-4737.

- - - . 1994. The human splicing factor ASF/SF2 can specifically recognize pre-mRNA 5′ splice sites. Proc. Natl. Acad. Sci. 91: 3363-3367.

Claims:

1. A protein having a splice factor activity in plants, characterized in that it
comprises the amino acid sequence of the protein according to FIG. 1A of the appendix, or
comprises the sequence of the amino acids 1 to 4, 7 to 19, 45 to 52, 111 to 116, and 149 to 153 of the protein according to FIG. 1A of the appendix and has more than 85% similarity with this protein, or
comprises more than 60% similarity with the splice proteins atSRp34/SR1 and SF2/ASF according to FIG. 2 of the appendix, wherein the G-rich sequence, which corresponds to the amino acids 85 to 113 of the atSRp34/SR1 protein is substituted by an S-rich sequence, or
corresponds to, or is derived from, the protein corresponding to FIG. 1A from a plant other than Arabidopsis thaliana.

2. A protein according to claim 1, characterized in that it comprises the sequence of the amino acids 1 to 4, 7 to 19, 22 to 72, 74 to 85, 96 to 141, 149 to 153, 156 to 172, with amino acid 168 being variable, yet not D or N, of the protein according to FIG. 1A of the appendix.

3. A protein according to claim 1 or 2, characterized in that it comprises an amino acid sequence having at least 90% identity with the sequence of the amino acids 1 to 85 and 96 to 222 of the protein according to FIG. 1A of the appendix.

4. A protein according to any one of claims 1 to 3, characterized in that it comprises an amino acid sequence that has at least 95%, in particular at least 98%, identity with the sequence of the amino acids 1 to 85 and 96 to 222 of the protein according to FIG. 1A of the appendix.

5. A nucleic acid molecule, characterized in that it
comprises a nucleic acid sequence according to FIG. 1A of the appendix, or
comprises a nucleic acid sequence encoding a protein according to any one of claims 1 to 4, or
comprises a nucleic acid sequence which, under stringent conditions, binds to the nucleic acid molecule according to FIG. 1A of the appendix and encodes a splice protein active in plants or is complementary thereto.

6. A biologically functional vector, characterized in that it comprises a nucleic acid molecule according to claim 5.

7. A system comprising a nucleic acid which encodes a protein according to any one of claims 1 to 4, and a nucleic acid which encodes
the atSRp34/SR1 protein of Arabidopsis thaliana or
the protein corresponding to FIG. 1A of the appendix from a plant other than Arabidopsis thaliana, or
a protein derived from these proteins, wherein at least one of the nucleic acids is under the control of a promoter not naturally connected with these nucleic acids.

8. A system according to claim 7, characterized in that both nucleic acids are controlled by promoters which are not naturally connected with these nucleic acids.

9. A system according to claim 7 or 8, characterized in that at least one of the promoters under whose control the nucleic acids are is an inducible promoter.

10. A system according to any one of claims 7 to 9, characterized in that the nucleic acid coding for a protein according to any one of claims 1 to 4 is under the control of a promoter which causes an overexpression of this protein.

11. A system according to any one of claims 7 to 10, characterized in that the nucleic acid which encodes a protein according to any one of claims 1 to 4 is under the control of a promoter which, under defined conditions, prevents the expression of this protein and, under defined other conditions, enables the expression of this protein.

12. A transgenic plant or plant cell, characterized in that it expresses a protein according to any one of claims 1 to 4.

13. A transgenic plant or plant cell, characterized in that it comprises a nucleic acid molecule according to claim 5 or a vector according to claim 6.

14. A transgenic plant or plant cell, characterized in that it comprises a system according to any one of claims 7 to 11.

15. The use of a protein according to any one of claims 1 to 4 of a nucleic acid according to claim 5, of a vector according to claim 6 or of a system according to any one of claims 7 to 11 for changing the splice properties of a plant cell or of a plant.

16. The use of a protein according to any one of claims 1 to 4, of a nucleic acid according to claim 5, of a vector according to claim 6 or of a system according to any one of claims 7 to 11 for changing the development behavior of a plant.

17. The use of a protein according to any one of claims 1 to 4, of a nucleic acid according to claim 5, of a vector according to claim 6 or of a system according to any one of claims 7 to 11 for retarding the flower formation of plants.

18. The use according to claim 17, characterised in that the retardation of the flower formation relative to the wild-type is at least 15%, preferably at least 25%.
